(19) United States
(12) Patent Application Publication
(10) Pub. No.: US 2020/0236969 A1
(43) Pub. Date: Jul. 30, 2020

(54) METHODS AND COMPOSITIONS FOR IMPROVING GROWTH AND PERFORMANCE

(71) Applicants: Timothy Johnson, Minneapolis, MN (US); Jessica Danzeisen, Minneapolis, MN (US); Bonnie Weber, Minneapolis, MN (US); REGENTS OF THE UNIVERSITY OF MINNESOTA, Minneapolis, MN (US)

(72) Inventors: Timothy Johnson, Richmond, MN (US); Jessica Danzeisen, Vadnais Heights, MN (US); Bonnie Weber, Inver Grove Heights, MN (US)

(73) Assignee: REGENTS OF THE UNIVERSITY OF MINNESOTA, Minneapolis, MN (US)

(21) Appl. No.: 16/756,675

(22) PCT Filed: Oct. 16, 2018
(86) PCT No.: PCT/US2018/056033

§ 371 Date: Apr. 16, 2020

Related U.S. Application Data
(60) Provisional application No. 62/572,732, filed on Oct. 16, 2017.

(51) Int. Cl. A23K 10/18; A23K 50/75; A61K 35/742; A61K 35/747

(52) U.S. Cl. CPC A23K 10/18; A23K 50/75; A61K 35/742; A61K 35/747; A23Y 2220/03; A23Y 2220/71; A23Y 2220/79; A23Y 2280/15; A23Y 2220/43

(57) ABSTRACT

Compositions including a bacterial species or a combination of bacterial species and methods of using those compositions are described. In some embodiments, use of the compositions prevents disease and/or promotes growth in an animal. In some embodiments, the animal is a turkey. In some embodiments, the composition is antibiotic-free.

### CONTINUING APPLICATION DATA

This application claims the benefit of U.S. Provisional Application Ser. No. 62/572,732, filed Oct. 16, 2017, which is incorporated by reference herein.

### SEQUENCE LISTING

This application contains a Sequence Listing electronically submitted to the United States Patent and Trademark Office via EFS-Web as an ASCII text file entitled “Sequence-Listing-110.05240201_ST25.txt” having a size of 56 kilobytes and created on Oct. 12, 2018. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### BACKGROUND

Because of concerns related to the use of antibiotics in animal agriculture, antibiotic-free alternatives are needed to prevent disease and promote animal growth. One of the current challenges facing commercial turkey production is declining performance as a result of reducing antimicrobial use.

### SUMMARY OF THE INVENTION

This disclosure describes methods and compositions for improving animal growth and performance. In one embodiment, this disclosure describes compositions including a bacterial species or a combination of bacterial species and methods of using those compositions. In some embodiments, use of those compositions prevents disease and/or promotes growth in an animal. In some embodiments, the animal is a turkey. In some embodiments, the composition is antibiotic-free. In one aspect, this disclosure describes a composition that includes at least 4 bacterial species or strains selected from the following bacterial species: Clostridium bartlettii; Lactobacillus acidophilus; Lactobacillus aviarius; Lactobacillus crispatus; Lactobacillus gallinarum; Lactobacillus helveticus; Lactobacillus ingluviei; Lactobacillus johnsonii; Lactobacillus reuteri; Lactobacillus salivarius; Lactobacillus vaginalis; and Pediococcus acidolactici. In some embodiments, at least one of the bacterial species or strains is an avian-sourced strain. In some embodiments, at least one of the bacterial species or strains is a turkey-sourced strain.

In another aspect, this disclosure describes a method that includes administering the composition to an animal. In some embodiments, the animal is a turkey. In some embodiments, the method further comprises administering a prebiotic to the animal.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (for example, 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows a phylogenetic tree of Lactobacillus johnsonii isolates. Turkey-source isolates are shown by gray lines, chicken-source isolates are shown by dotted lines, pig-source isolates are shown by dashed lines, and human/rodent-source isolates are shown by dotted/dashed lines. Turkey-source (gray lines) isolates were further subdivided into clades 1-8 based upon their single-nucleotide polymorphism (SNP) and gene content differences. The scale bar in the lower left indicates a scale of 4000 SNPs.

FIG. 2 shows body weights in grams (y-axis) of day-of-hatch turkeys inoculated with five different L. johnsonii isolates, compared with a saline (negative control) inoculum. Days are presented in age of turkeys. Error bars depict standard error of means. Letters depict groups based on statistical significance using ANOVA with Tukey's adjustment (P<0.05); “a” is significantly different from “b.” For each treatment group, five replicate cages were used with 10 birds per cage (n=50 per treatment group).

FIG. 3 shows a phylogenetic tree of Lactobacillus aviarius isolates. Turkey-source isolates of L. aviarius subsp. aviarius are shown by gray lines, and chicken-source isolates of L. aviarius subsp. aviarius and L. aviarius subsp. araffinosus are shown by dotted lines.

FIG. 4A and FIG. 4B show average body weights in grams (y-axis) for day-of-hatch turkeys treated with a 4-strain probiotic blend, a 10-strain (derived from birds aged 3 weeks) probiotic blend, and a 10-strain (derived from birds aged 6 weeks) probiotic blend, as described in Example 2, compared to body weights for day-of-hatch turkeys treated with a saline negative control and day-of-hatch turkeys treated with an existing commercial probiotic derived from chickens (FM-B11). FIG. 4A. Turkeys were weighed on day 0 (that is, day-of-hatch), day 7, day 10, and day 14. Error bars depict standard error of means. Letters depict groups based on statistical significance using ANOVA (P<0.05); “a” is significantly different from “b,” and “ab” is not significantly different from either “a” or “b”. FIG. 4B shows average body weights in grams (y-axis) for day 14 turkeys with an adjusted y-axis. For each treatment group, five replicate cages were used with 10 birds per cage (n=50 per treatment group).

FIG. 5A and FIG. 5B show average body weights in grams (y-axis) for day-of-hatch turkeys treated with a 4-strain probiotic blend inoculation group, day-of-hatch turkeys treated with an existing commercial probiotic derived from chickens (FM-B11), and day-of-hatch turkeys treated with a low-dose antibiotic (bacitracin methylene disalicylate or BMD), as described in Example 2, compared to day-of-hatch turkeys treated with a saline negative control. FIG. 5A. Turkeys were weighed on day 0 (that is, day-of-hatch), day 3, day 6, and day 13. Error bars depict standard error of means. Letters depict groups based on statistical significance using ANOVA with Tukey's adjustment (P<0.05); “a” is significantly different from “b,” and “ab” is not significantly different from either “a” or “b”. FIG. 5B shows average body weights in grams (y-axis) for day 13 turkeys with an adjusted y-axis. For each treatment group, eight replicate cages were used with 10 birds per cage (n=80 per treatment group).

### DETAILED DESCRIPTION OF ILLUSTRATIVE EMBODIMENTS

This disclosure describes compositions including a bacterial species or a combination of bacterial species and methods of using those compositions. Alternatives to antimicrobials such as antibiotics exist for the broiler chicken industry, but such alternatives are lacking for the commercial turkey industry. Moreover, many alternatives to antimicrobials are aimed at pathogen reduction and not performance. In some embodiments, the compositions disclosed herein prevent disease and/or promote growth in an animal.

In some embodiments, the animal is preferably a bird. In some embodiments, the animal is preferably a turkey.

### Compositions

The compositions of the present disclosure include a bacterial species or a combination of bacterial species. In some embodiments, as further described below, a composition includes a combination of specific bacterial species or strains.

In some embodiments, the composition is preferably antibiotic-free.

Bacterial Species and/or Strains in the Composition

In some embodiments, the composition includes at least 4 bacterial species or strains, at least 5 bacterial species or strains, at least 6 bacterial species or strains, at least 7 bacterial species or strains, at least 8 bacterial species or strains, at least 9 bacterial species or strains, at least 10 bacterial species or strains, at least 11 bacterial species or strains, or at least 12 bacterial species or strains selected from the following bacterial species: Clostridium (Intestinibacter) bartlettii; Lactobacillus acidophilus; Lactobacillus aviarius; Lactobacillus crispatus; Lactobacillus gallinarum; Lactobacillus helveticus; Lactobacillus ingluviei; Lactobacillus johnsonii; Lactobacillus reuteri; Lactobacillus salivarius; Lactobacillus vaginalis; and Pediococcus acidilactici.

In some embodiments, at least one of the bacterial species or strains may have been isolated from an avian source (that is, is avian-sourced). In some embodiments, at least 2 bacterial species or strains, at least 3 bacterial species or strains, at least 4 bacterial species or strains, or all of the bacterial species or strains are from an avian source.

In some embodiments, at least one of the bacterial species or strains may have been isolated from a turkey (that is, is turkey-sourced). In some embodiments, at least 2 bacterial species or strains, at least 3 bacterial species or strains, at least 4 bacterial species or strains, or all of the bacterial species or strains are from a turkey.

In some embodiments, the composition includes a strain of Clostridium bartlettii. In some embodiments, the strain of Clostridium bartlettii includes Clostridium bartlettii DSM 16795 (deposited in the NCBI database under GENBANK accession number FUXV00000000.1).

In some embodiments, the composition includes a strain of Lactobacillus acidophilus. In some embodiments, a strain of Lactobacillus acidophilus includes Lactobacillus acidophilus strain UMNPBX11 (deposited in the NCBI database under Bioproject accession number PRJNA412075 and GENBANK accession number PCYZ00000000).

In some embodiments, the composition includes a strain of Lactobacillus aviaries. In some embodiments, a strain of Lactobacillus aviarius includes at least one of Lactobacillus aviarius subspecies araffinosus and Lactobacillus aviarius subspecies aviaries. In some embodiments, a strain of Lactobacillus acidophilus includes at least one of Lactobacillus aviarius strain UMNLav12 (deposited in the NCBI database under GENBANK accession number NZ_LWUE00000000.1); Lactobacillus aviarius strain UMNLav13 deposited in the NCBI database under GENBANK accession number NZ_LWUF00000000.1); Lactobacillus aviarius strain UMNLAv76 (deposited in the NCBI database under GENBANK accession number PCZQ00000000); Lactobacillus aviarius strain UMNLav97 (deposited in the NCBI database under GENBANK accession number PCZP00000000); Lactobacillus aviarius strain UMNLav98 (deposited in the NCBI database under GENBANK accession number PCZO00000000); Lactobacillus aviarius strain DSM 20653 (deposited in the NCBI database under GENBANK accession number NZ_AYYZ00000000.1); and Lactobacillus aviarius strain DSM 20655 (deposited in the NCBI database under GENBANK accession number NZ_AYZA00000000.1).

In some embodiments, the composition includes a strain of Lactobacillus crispatus. In some embodiments, a strain of Lactobacillus crispatus includes at least one of Lactobacillus crispatus strain UMNPBX1 (deposited in the NCBI database under GENBANK accession number PCZI00000000); Lactobacillus crispatus strain UMNPBX8 (deposited in the NCBI database under GENBANK accession number PCZB00000000); Lactobacillus crispatus strain UMNPBX12 (deposited in the NCBI database under GENBANK accession number PCYY00000000); Lactobacillus crispatus strain UMNPBX15 (deposited in the NCBI database under GENBANK accession number PCYV00000000); and Lactobacillus crispatus strain UMNPBX16 (deposited in the NCBI database under GENBANK accession number PCYU00000000).

In some embodiments, the composition includes a strain of Lactobacillus gallinarum. In some embodiments, a strain of Lactobacillus gallinarum includes at least one of Lactobacillus gallinarum strain UMNPBX4 (deposited in the NCBI database under GENBANK accession number PCZF00000000); Lactobacillus gallinarum strain UMNPBX14 (deposited in the NCBI database under GENBANK accession number PCYW00000000); and Lactobacillus gallinarum strain UMNPBX17 (deposited in NCBI databases under GENBANK accession number PCYT00000000).

In some embodiments, the composition includes a strain of Lactobacillus helveticus. In some embodiments, a strain of Lactobacillus helveticus includes Lactobacillus helveticus strain UMNPBX6 (deposited in the NCBI database under GENBANK accession number PCZD00000000).

In some embodiments, the composition includes Lactobacillus ingluviei. In some embodiments, a strain of Lactobacillus ingluviei includes Lactobacillus ingluviei strain UMNPBX19 (deposited in the NCBI database under GENBANK accession number PCYR00000000).

In some embodiments, the composition includes a strain of Lactobacillus johnsonii. In some embodiments, a strain of Lactobacillus johnsonii includes at least one of Lactobacillus johnsonii strain UMNLJ21 (deposited in the NCBI database under GENBANK accession numbers CP021701, CP021702, and CP021703); Lactobacillus johnsonii strain UMNLJ94 (deposited in the NCBI database under GENBANK accession number PCZK00000000); and Lactobacillus johnsonii strain UMNLJ113 (deposited in the NCBI database under GENBANK accession number PCZJ00000000).

In some embodiments, the composition includes a strain of Lactobacillus reuteri. In some embodiments, a strain of Lactobacillus reuteri includes at least one of Lactobacillus reuteri strain UMNPBX3 (deposited in the NCBI database under GENBANK accession number PCZG00000000); Lactobacillus reuteri strain UMNPBX7 (deposited in the NCBI database under GENBANK accession number PCZC00000000); Lactobacillus reuteri strain UMNPBX10 (deposited in the NCBI database under GENBANK accession number PCZL00000000); and Lactobacillus reuteri strain UMNPBX18 (deposited in the NCBI database under GENBANK accession number PCYS00000000).

In some embodiments, the composition includes a strain of Lactobacillus salivarius. In some embodiments, a strain of Lactobacillus salivarius includes at least one of Lactobacillus salivarius strain UMNPBX2 (deposited in the NCBI database under GENBANK accession number PCZH00000000); and Lactobacillus salivarius strain UMNPBX9 (deposited in the NCBI database under GENBANK accession number PCZA00000000).

In some embodiments, the composition includes a strain of Lactobacillus vaginalis. In some embodiments, a strain of Lactobacillus vaginalis includes at least one of Lactobacillus vaginalis strain UMNPBX5 (deposited in the NCBI database GENBANK accession number PCZE00000000); and Lactobacillus vaginalis strain UMNPBX13 (deposited in the NCBI database under GENBANK accession number PCYX00000000).

In some embodiments, the composition includes a strain of Pediococcus acidolactici. In some embodiments, a strain of Pediococcus acidolactici includes Pediococcus acidolactici UMNPBX20 (deposited in the NCBI database under GENBANK accession number PCYQ00000000).

Although the strains described herein are, in some embodiments, described by reference to particular sequences, a person having skill in the art will recognize that a sequence having some genetic variations from a sequence provided for the strain described herein will still describe the same strain. In some embodiments, a sequence differing by fewer than 20,000 SNPs, fewer than 10,000 SNPs, fewer than 5,000 SNPs, fewer than 1,000 SNPs, fewer than 750 SNPs, fewer than 500 SNPs, fewer than 400 SNPs, fewer than 300 SNPs, fewer than 200 SNPs, or fewer than 100 SNPs from the sequence of a strain described herein is defined as describing the same strain. In some embodiments, a sequence differing by less than 0.5%, less than 0.4%, less than 0.3%, less than 0.2%, or less than 0.1% from the sequence of a strain described herein is defined as describing the same strain. Percent identity between two sequences (for example, nucleic acid sequences or amino acids sequences) may be determined in any one of numerous ways known to a person having skill in the art including, for example, using publicly available computer software such as the Smith-Waterman algorithm, BESTFIT alignment, BLAST, FASTA, CLUSTALW, etc.

### Concentration of the Bacteria in the Composition

In some embodiments, the composition includes at least 1×10⁵ colony-forming units (CFU) of each of the bacterial species or strains, at least 1×10⁶ CFU of each of the species or strains, at least 1×10⁷ CFU of each of the species or strains, or at least 1×10⁸ CFU of each of the species or strains. In some embodiments, the composition includes up to 1×10⁶ CFU of each of the species or strains, up to 1×10⁷ CFU each of the species or strains, up to 1×10⁸ CFU each of the species or strains, up to 1×10⁹ CFU each of the species or strains, up to 1×10¹⁰ CFU each of the species or strains, or up to 1λ10¹¹ CFU each of the species or strains. For example, in some embodiments, a composition may include at least 1×10⁷ CFU of each of the species or strains and up to 1×10⁹ CFU of each of the species or strains. In some embodiments, a composition may include 1×10⁸ CFU of each of the species or strains.

In some embodiments, a viability count may be used to determine the number of CFU of the composition.

### Methods of Using the Compositions

The compositions described herein may be administered to an animal using any suitable method. In some embodiments, the composition may be administered as a feed additive. In some embodiments, the composition may be administered by oral gavage. In some embodiments, the composition may be administered in water and/or in a nutrient gel. In some embodiments, the composition may be micro-encapsulated.

In some embodiments, the composition may be administered multiple times. For example, the composition may be administered at least twice, at least three times, or at least four times. In some embodiments, the composition may be administered up to four times, up to five times, up to 10 times, up to 15 times, or up to 18 times. In some embodiments, the composition may be administered on multiple days. In some embodiments the composition may be administered weekly.

In some embodiments, the composition may be administered daily and/or continuously, for example, in feed. In some embodiments, the administration of the composition may be timed to coincide with an event in the animal's development or treatment. For example, the composition may be administered in combination with one or more of the following events: before and/or after vaccination, before and/or after a move of the animal; before and/or after a diet change, before and/or after diagnosis with a disease, and before and/or after treatment for a disease.

In some embodiments, the composition may be administered to an animal that is less than a week old. For example, in some embodiments, the composition may be administered to a day-of-hatch bird. In some embodiments, the animal may be at least one week old, at least two weeks old, at least three weeks old, or at least four weeks old.

In some embodiments, the composition may be administered to an animal in combination with a prebiotic. The prebiotic may be administered by any suitable method. In some embodiments, a prebiotic may be administered at the same time—including, for example, in the composition that includes a combination of bacterial species or strains—or at a different time or different times from the composition. In some embodiments, a prebiotic may be administered continuously to the animal. In some embodiments, as described, for example, in Example 3, the prebiotic may be included in the animal's feed. The prebiotic may include any suitable prebiotic including, for example, one or more of a disaccharide, an oligosaccharide, and a polysaccharide. For example, a prebiotic may include one or more of lactose, lactulose, mannan-oligosaccharide (MOS), inulin, soy Fructo-oligosaccharide (FOS), galacto-oligosaccharide (GOS), and a β-glucan. In some embodiments, the prebiotic may preferably include lactose. In some embodiments, the prebiotic may preferably include a mannan-oligosaccharide, and/or a β-glucan. In some embodiments, the prebiotic may include SAFMANNAN (available from Phileo Lesaffre Animal Care, Marcq-En-Baroeul Cedex, France).

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Approximately 100 different hybrid turkeys ranging in age from 0-12 weeks were used to collect bacterial strains. Upon humane euthanization, the ileum was aseptically removed from each turkey and contents from each ileum section were homogenized using a stomacher. Ten-fold serial dilutions of each homogenized sample were performed in phosphate-buffered saline. Samples were then plated onto three types of media: 1) Lactobacillus selection (LBS) agar (BD Diagnostics Systems, Hunt Valley, Md.), 2) Lactobacilli MRS agar (BD Diagnostic Systems, Hunt Valley, Md.), and 3) Trypticase Soy Agar with 5% sheep blood (BD Diagnostic Systems, Hunt Valley, Md.). Plates were incubated both aerobically and anaerobically overnight at 37° C. Isolated colonies were then selected from each plate and stored for further use in 20 percent weight-volume (% w:v) glycerol. In total, 1,267 isolates were obtained.

Each isolate was identified at the bacterial species level using sequencing of the full length bacterial 16S rRNA, as previously described (Lane DJ, 1991, 16S/23S rRNA sequencing. In: E. Stackebrandt, M. Goodfellow. Nucleic acid techniques in bacterial systematics. New York, N.Y., John Wiley & Sons, Inc., pp. 115-176.). Following species-level identification of these isolates, representative isolates from each bacterial species were subjected to whole genome sequencing using Illumina MiSeq at a depth of at least 50× coverage (Illumina, Inc., San Diego, Calif.).

For L. johnsonii and L. aviarius, each isolate was mapped to a reference genome matching its respective bacterial species using CLC Genomics Workbench (Qiagen, N.V., Hilden, Germany). Following mapping, single nucleotide polymorphisms (SNPs) were identified using variant calling in CLC Genomics Workbench. SNPs were then combined for each bacterial species of interest, and subsequently analyzed. Using Maximum Parsimony methods (MEGA, available at on the world wide web at megasoftware.net, version 6.06), a phylogenetic tree was constructed to group isolates based upon genetic similarity and to establish clades within each bacterial species. Finally, a pangenome analysis was performed for every isolate within each clade of a species, to establish core and unique genes belonging to each subset; an exemplary process is described in Example 1A.

For other bacterial species, strains were selected based upon their dominance in high-performing turkey flocks and upon the isolation of the strains from commercial turkeys of certain age groups.

### Example 1A—Selection of Lactobacillus johnsonii Strain UMNLJ21

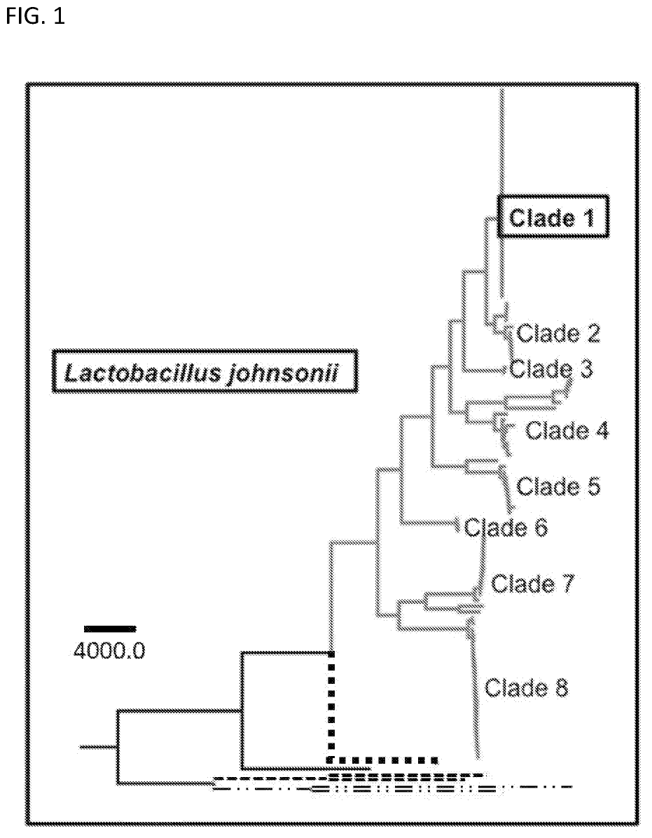

One hundred seventeen L. johnsonii isolates were analyzed as described above. SNPs were used for phylogenetic analysis including all L. johnsonii isolates in the NCBI database (February 2017). Using Maximum Parsimony methods (MEGA, available at on the world wide web at megasoftware.net, version 6.06) a phylogenetic tree was constructed using the following non-turkey species sequences: chicken strain FI9785, GENBANK accession NC_013504; human strain NCC 533, GENBANK accession NC_005362; pig strain BS15, GENBANK accession NZ_CP016400; pig strain DPC 6026, GENBANK accession NC_017477; human strain N6.2, GENBANK accession NC_022909. Isolates were separated into clades based upon their host source of isolation (human, rodent, pig, chicken, or turkey). Furthermore, eight distinct clades were identified among the 117 turkey-source isolates (FIG. 1). These clades were used for further study.

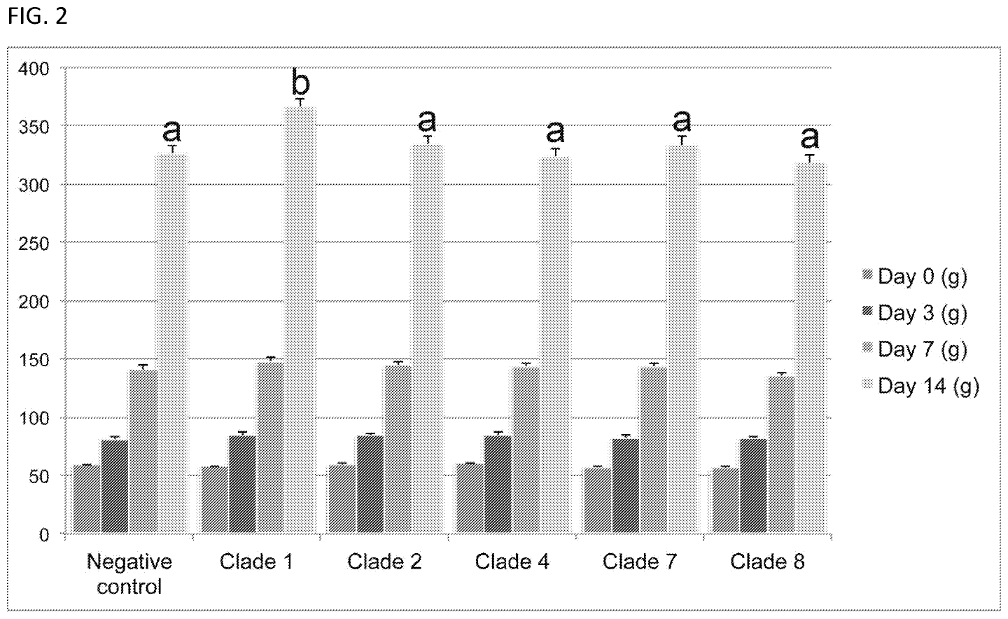

Strains were selected from each major clade and tested for their ability to enhance turkey growth. In this experiment, day-of-hatch turkey poults (n=360 total) in a caged trial with 6 replicate cages per treatment group (n=60 per treatment) were inoculated via oral gavage with either a negative saline control (negative control) or 1λ10⁹ colony forming units (CFU) of L. johnsonii belonging to one of five clades (Clades 1, 2, 4, 7, and 8, see FIG. 1). Birds were weighed at four time points. Only the Clade 1 isolate was able to significantly enhance body weight in this experiment (P<0.01). Results are shown in FIG. 2 and Table 1. These results indicated that Clade 1 isolates have the propensity to enhance performance in commercial turkeys.

TABLE 1
Average body weights for turkey poults administered single strain
L. johnsonii combinations. SEM = standard error of means.

| | Day 0 | Day 0 | Day 3 | Day 3 | Day 7 | Day 7 | Day 14 | Day 14 |
|---|---|---|---|---|---|---|---|---|
| | (g) | SEM | (g) | SEM | (g) | SEM | (g) | SEM |
| Negative control | 59.2 | 1.3 | 81.3 | 2.5 | 141.1 | 2.3 | 326.5 | 10.9 |
| Clade 1 | 57.7 | 1.3 | 84.8 | 2.5 | 148.1 | 2.3 | 366.5 | 10.9 |
| Clade 2 | 59.3 | 1.3 | 84.4 | 2.5 | 144.4 | 2.3 | 334.7 | 10.9 |
| Clade 4 | 60.1 | 1.3 | 84.6 | 2.5 | 143.2 | 2.3 | 324 | 10.9 |
| Clade 7 | 57.2 | 1.3 | 82 | 2.5 | 143.3 | 2.3 | 333.5 | 10.9 |
| Clade 8 | 56.7 | 1.3 | 81.5 | 2.5 | 135.5 | 2.3 | 318.6 | 10.9 |

Pangenome comparisons identified genes that were unique to Clade 1 isolates. A total of 49 genes were identified that were conserved across Clade 1 isolates, but unique to Clade 1 isolates compared to all other L. johnsonii. A list of the genes and their predicted functions are provided in Table 2; corresponding sequences of the genes of Table 2 are provided as SEQ ID NOs:1-49. These genes included those predicted to encode for ABC transport systems (predicted for novel sugar utilization), CRISPR/Cas system (for bacterial defense against foreign DNA), mucus-binding proteins (for colonization), exopolysaccharide production (for survival and colonization), and restriction modification systems (for bacterial defense against foreign DNA). Thus, this gene subset includes unique proteins that may encode for the beneficial growth properties conferred in commercial turkeys.

The genome sequence of this strain is available in the NCBI database under accession numbers CP021701, CP021702, and CP021703. Clade 1 isolates differ from their closest clade (clade 2) by at least 2,921 SNPs, and differ from a chicken-source isolate described in the literature for its probiotic properties in broiler chickens (PMID Nos. 28318296, 19767436, 14962040) by 20,509 SNPs.

TABLE 2
List of unique genes present in clade 1 of
L. johnsonii and their predicted functions.

| Gene | | |
|---|---|---|
| locus | Sequence | SEQ ID NO |
| 79 | YSIRK signal domain/LPXTG anchor domain | SEQ ID NO: 1 |
| | surface protein | |
| 100 | hypothetical protein | SEQ ID NO: 2 |
| 113 | Type II restriction/modification system, DNA | SEQ ID NO: 3 |
| | methylase subunit YeeA | |
| 269 | DNA (cytosine-5-)-methyltransferase | SEQ ID NO: 4 |
| 386 | putative antibiotic resistance protein | SEQ ID NO: 5 |
| 417 | ABC transporter, ATP-binding cassette protein | SEQ ID NO: 6 |
| 517 | Type I restriction modification DNA | SEQ ID NO: 7 |
| | specificity protein | |
| 584 | putative polysaccharide biosynthesis protein | SEQ ID NO: 8 |
| 609 | hypothetical protein | SEQ ID NO: 9 |
| 877 | putative glycosyltransferase | SEQ ID NO: 10 |
| 921 | putative DNA-binding protein | SEQ ID NO: 11 |
| 946 | Type II restriction enzyme HphI | SEQ ID NO: 12 |
| 968 | putative glycosyltransferase | SEQ ID NO: 13 |
| 979 | putative polysaccharide biosynthesis protein | SEQ ID NO: 14 |
| 1088 | cell division protein FtsK | SEQ ID NO: 15 |
| 1104 | putative replication initiation protein | SEQ ID NO: 16 |
| 1133 | cell division protein | SEQ ID NO: 17 |
| 1134 | hypothetical protein | SEQ ID NO: 18 |
| 1152 | putative glycosyltransferase | SEQ ID NO: 19 |
| 1160 | site-specific integrase | SEQ ID NO: 20 |
| 1180 | putative sulfotransferase | SEQ ID NO: 21 |
| 1350 | putative glycosyltransferase | SEQ ID NO: 22 |
| 1537 | hypothetical protein | SEQ ID NO: 23 |
| 1569 | DNA methyltransferase | SEQ ID NO: 24 |
| 1638 | hypothetical protein | SEQ ID NO: 25 |
| 1696 | hypothetical protein | SEQ ID NO: 26 |
| 1726 | putative glycosyltransferase | SEQ ID NO: 27 |
| 2099 | hypothetical protein | SEQ ID NO: 28 |
| 2228 | hypothetical protein | SEQ ID NO: 29 |
| 2447 | type II-A CRISPR-associated protein Csn2 | SEQ ID NO: 30 |
| 2469 | hypothetical protein | SEQ ID NO: 31 |
| 2499 | putative membrane protein | SEQ ID NO: 32 |
| 2502 | IS110 transposase | SEQ ID NO: 33 |
| 2529 | ISL3 transposase | SEQ ID NO: 34 |
| 3071 | hypothetical protein | SEQ ID NO: 35 |
| 3106 | bacteriophage infection resistance protein | SEQ ID NO: 36 |
| 3140 | hypothetical protein | SEQ ID NO: 37 |
| 3220 | hypothetical protein | SEQ ID NO: 38 |
| 3641 | hypothetical protein | SEQ ID NO: 39 |
| 3725 | hypothetical protein | SEQ ID NO: 40 |
| 3836 | hypothetical protein | SEQ ID NO: 41 |
| 3879 | hypothetical protein | SEQ ID NO: 42 |
| 3880 | transposase | SEQ ID NO: 43 |
| 3963 | hypothetical protein | SEQ ID NO: 44 |
| 3979 | hypothetical protein | SEQ ID NO: 45 |
| 4005 | hypothetical protein | SEQ ID NO: 46 |
| 4021 | hypothetical protein | SEQ ID NO: 47 |
| 4040 | hypothetical protein | SEQ ID NO: 48 |
| 4044 | hypothetical protein | SEQ ID NO: 49 |

### Example 1B—Lactobacillus aviarius

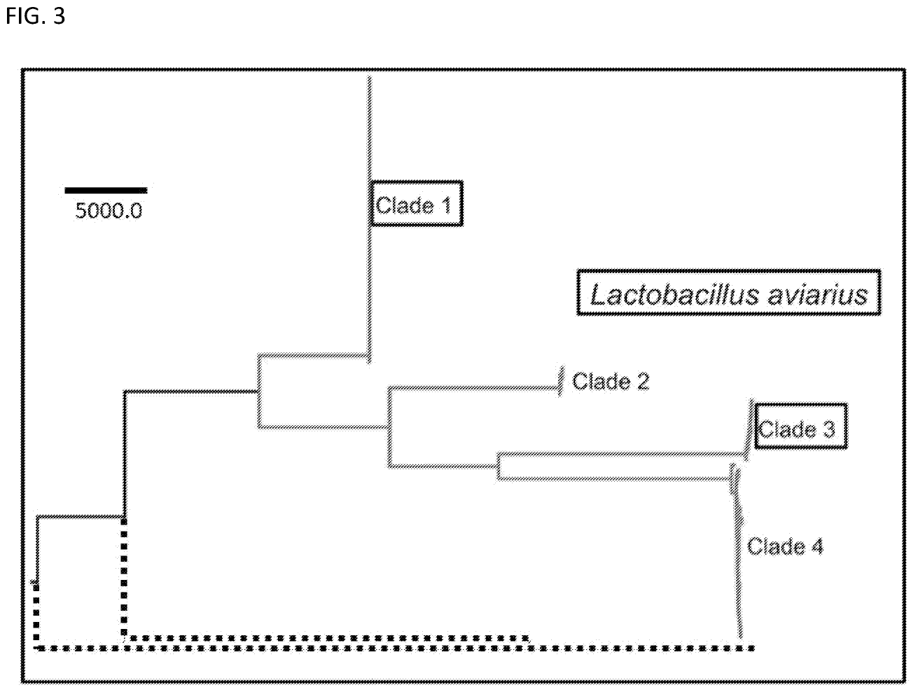

One hundred and four L. aviarius isolates were analyzed as described above. SNPs were used for phylogenetic analysis including all L. aviarius isolates in the NCBI database (February 2017). Using Maximum Parsimony methods, a phylogenetic tree was constructed. Isolates were separated into clades based upon their host source of isolation (chicken or turkey). Furthermore, distinct clades were identified among the 104 turkey-source isolates differentiating L. aviarius subsp. aviarius isolates (FIG. 3).

Two strains from clades 1 and 4 were selected for further study (see Example 2 (4-strain probiotic blend)). Strains selected included UMNLav12 (NCBI Biosample SAMN04573032) and UMNLav13 (NCBI Biosample AMN04573033). These strains differ from chicken source L. aviarius by at least 42,318 SNPs. Also, each clade-associated strain within turkeys differs from strains of other clades by at least 31,264 SNPs.

### Example 1C—Clostridium bartlettii (or Intestinibacter bartlettii)

A strain of Clostridium bartlettii was isolated from a 6-week-old commercial turkey.

### Example 2

The following combinations of bacterial species and/or strains were tested. Additional information about the strains in each blend is provided in Tables 3A, 3B, 3C, and 3D, respectively.

### 4-Strain Probiotic Blend:

Clostridium bartlettii strain DSM 16795 (deposited in the NCBI database under Bioproject accession number PRJNA245632 and GENBANK accession number FUXV00000000.1)
Lactobacillus aviarius strain UMNLav12 (deposited in the NCBI database under Bioproject accession number PRJNA316009 and GENBANK accession number NZ_LWUE00000000.1)
Lactobacillus aviarius strain UMNLav13 (deposited in the NCBI database under Bioproject accession number and GENBANK accession number NZ_LWUF00000000.1)
Lactobacillus johnsonii strain UMNLJ21 (deposited in the NCBI database under Bioproject accession number PRJNA316010 and GENBANK accession numbers CP021701, CP021702, and CP021703)

### 10-Strain (3 Week) Probiotic Blend:

Lactobacillus crispatus strain UMNPBX1 (deposited in the NCBI database under Bioproject accession number PRJNA412075 and GENBANK accession number PCZI00000000)
Lactobacillus crispatus strain UMNPBX8 (deposited in the NCBI database under Bioproject accession number PRJNA412075 and GENBANK accession number PCZB00000000)
Lactobacillus salivarius strain UMNPBX2 (deposited in the NCBI database under Bioproject accession number PRJNA412075 and GENBANK accession number PCZH00000000)
Lactobacillus reuteri strain UMNPBX3 (deposited in the NCBI database under Bioproject accession number PRJNA412075 and GENBANK accession number PCZG00000000)
Lactobacillus gallinarum strain UMNPBX4 (deposited in the NCBI database under Bioproject accession number PRJNA412075 and GENBANK accession number PCZF00000000)
Lactobacillus vaginalis strain UMNPBX5 (deposited in the NCBI database under Bioproject accession number PRJNA412075 and GENBANK accession number PCZE00000000)
Lactobacillus helveticus strain UMNPBX6 (deposited in the NCBI database under Bioproject accession number PRJNA412075 and GENBANK accession number PCZD00000000)
Lactobacillus johnsonii strain UMNLJ113 (deposited in the NCBI database under Bioproject accession number PRJNA316010 and GENBANK accession number PCZJ00000000)
Lactobacillus aviarius strain UMNLAv76 (deposited in the NCBI database under Bioproject accession number PRJNA316009 and GENBANK accession number PCZQ00000000)
Lactobacillus reuteri strain UMNPBX7 (deposited in the NCBI database under Bioproject accession number PRJNA412075 and GENBANK accession number PCZC00000000)

### 10-Strain (6 Week) Probiotic Blend:

Lactobacillus salivarius strain UMNPBX9 (deposited in the NCBI database under Bioproject accession number PRJNA412075 and GENBANK accession number PCZA00000000)
Lactobacillus reuteri strain UMNPBX10 (deposited in the NCBI database under Bioproject accession number PRJNA412075 and GENBANK accession number PCZL00000000)
Lactobacillus acidophilus strain UMNPBX11 (deposited in the NCBI database under Bioproject accession number PRJNA412075 and GENBANK accession number PCYZ00000000)
Lactobacillus crispatus strain UMNPBX12 (deposited in the NCBI database under Bioproject accession number PRJNA412075 and GENBANK accession number PCYY00000000)
Lactobacillus vaginalis strain UMNPBX13 (deposited in the NCBI database under Bioproject accession number PRJNA412075 and GENBANK accession number PCYX00000000)
Lactobacillus gallinarum strain UMNPBX14 (deposited in the NCBI database under Bioproject accession number PRJNA412075 and GENBANK accession number PCYW00000000)
Lactobacillus crispatus strain UMNPBX15 (deposited in the NCBI database under Bioproject accession number PRJNA412075 and GENBANK accession number PCYV00000000)
Lactobacillus johnsonii strain UMNLJ21 (deposited in the NCBI database under Bioproject accession number PRJNA316010 and GENBANK accession numbers CP021701, CP021702, and CP021703)
Lactobacillus aviarius strain UMNLav97 (deposited in the NCBI database under Bioproject accession number PRJNA316009 and GENBANK accession number PCZP00000000)
Lactobacillus aviarius strain UMNLav98 (deposited in the NCBI database under Bioproject accession number PRJNA316009 and GENBANK accession number PCZO00000000)

### 12-Strain (Wild Card) Probiotic Blend:

Clostridium bartlettii strain DSM 16795 (deposited in NCBI databases under Bioproject accession number PRJNA245632 and GENBANK accession number FUXV00000000.1)
Lactobacillus aviarius strain UMNLAv97 (deposited in NCBI databases under Bioproject accession number PRJNA316009 and GENBANK accession number PCZP00000000)
Lactobacillus aviarius strain UMNLAv98 (deposited in NCBI databases under Bioproject accession number PRJNA316009 and GENBANK accession number PCZO00000000)
Lactobacillus aviarius strain DSM 20653 (deposited in NCBI databases under Bioproject accession number PRJNA222257 and GENBANK accession number NZ_AYYZ00000000.1)
Lactobacillus aviarius strain DSM 20655 (deposited in NCBI databases under Bioproject accession number PRJNA222257 and GENBANK accession number NZ_AYZA00000000.1)
Lactobacillus crispatus strain UMNPBX16 (deposited in NCBI databases under Bioproject accession number PRJNA412075 and GENBANK accession number PCYU00000000)
Lactobacillus gallinarum strain UMNPBX17 (deposited in NCBI databases under Bioproject accession number PRJNA412075 and GENBANK accession number PCYT00000000)
Lactobacillus ingluviei strain UMNPBX19 (deposited in NCBI databases under Bioproject accession number PRJNA412075 and GENBANK accession number PCYR00000000)
Lactobacillus johnsonii strain UMNLJ94 (deposited in NCBI databases under Bioproject accession number PRJNA316010 and GENBANK accession number PCZK00000000)
Lactobacillus johnsonii strain UMNLJ21 (deposited in NCBI databases under Bioproject accession number PRJNA316010 and GENBANK accession numbers CP021701, CP021702, and CP021703)
Lactobacillus reuteri strain UMNPBX18 (deposited in the NCBI database under Bioproject accession number PRJNA412075 and GENBANK accession number PCYS00000000)
Pediococcus acidilactici strain UMNPBX20 (deposited in the NCBI database under Bioproject accession number PRJNA412075 and GENBANK accession number PCYQ00000000)

TABLE 3A
4-strain combination

| | Host | | | | | |
|---|---|---|---|---|---|---|
| Target Strain | source | Strain name | Strain ID (internal) | Bioproject | Biosample | Genbank Accession |
| Lactobacillus johnsonii | Turkey | UMNLJ21 | MO-F2-W2-B3-C5 | PRJNA316010 | SAMN04573145 | CP021701, CP021702, |
| | | | | | | and CP021703 |
| Clostridium bartlettii | Human | DSM 16795 | ATCC Strain | PRJNA245632 | SAMN02745147 | FUXV00000000.1 |
| Lactobacillus aviarius | Turkey | UMNLAv12 | SNF2W2B1L1 | PRJNA316009 | SAMN04573032 | NZ_LWUE00000000.1 |
| Lactobacillus aviarius | Turkey | UMNLAv13 | SNF2W2B1L7 | PRJNA316009 | SAMN04573033 | NZ_LWUF00000000.1 |

TABLE 3B
10-strain combination from 3-wk-old birds

| | Host | | | | | |
|---|---|---|---|---|---|---|
| Target Strain | source | Strain name | Strain ID (internal) | Bioproject | Biosample | Genbank Accession |
| Lactobacillus crispatus | Turkey | UMNPBX1 | MO-F2-W3-B1-T2 | PRJNA412075 | SAMN07702352 | PCZI00000000 |
| Lactobacillus salivarius | Turkey | UMNPBX2 | MO-F2-W3-B1-T3 | PRJNA412075 | SAMN07702353 | PCZH00000000 |
| Lactobacillus reuteri | Turkey | UMNPBX3 | MO-F2-W3-B1-C8 | PRJNA412075 | SAMN07702354 | PCZG00000000 |
| Lactobacillus gallinarum | Turkey | UMNPBX4 | MO-F2-W3-B5-T3 | PRJNA412075 | SAMN07702355 | PCZF00000000 |
| Lactobacillus vaginalis | Turkey | UMNPBX5 | SNF2W3B1L7 | PRJNA412075 | SAMN07702356 | PCZE00000000 |
| Lactobacillus helveticus | Turkey | UMNPBX6 | SNF2W3B1L8 | PRJNA412075 | SAMN07702357 | PCZD00000000 |
| Lactobacillus johnsonii | Turkey | UMNLJ113 | SNF2W3B3L6 | PRJNA316010 | SAMN04573237 | PCZJ00000000 |
| Lactobacillus aviarius | Turkey | UMNLAv76 | SNF2W3B1M8 | PRJNA316009 | SAMN04573096 | PCZQ00000000 |
| Lactobacillus reuteri | Turkey | UMNPBX7 | SNF2W3B2M2 | PRJNA412075 | SAMN07702358 | PCZC00000000 |
| Lactobacillus crispatus | Turkey | UMNPBX8 | SNF2W3B2M1 | PRJNA412075 | SAMN07702359 | PCZB00000000 |

TABLE 3C
10-strain combination from 6-wk-old birds

| | Host | | | | | |
|---|---|---|---|---|---|---|
| Target Strain | source | Strain name | Strain ID (internal) | Bioproject | Biosample | Genbank Accession |
| Lactobacillus salivarius | Turkey | UMNPBX9 | MO-F2-W6-B4-C3 | PRJNA412075 | SAMN07702360 | PCZA00000000 |
| Lactobacillus johnsonii | Turkey | UMNLJ21 | MO-F2-W2-B3-C5 | PRJNA316010 | SAMN04573145 | CP021701, CP021702, |
| | | | | | | and CP021703 |
| Lactobacillus reuteri | Turkey | UMNPBX10 | MO-F2-W6-B4-C7 | PRJNA412075 | SAMN07702361 | PCZL00000000 |
| Lactobacillus acidophilus | Turkey | UMNPBX11 | MO-F2-W6-B4-C8 | PRJNA412075 | SAMN07702362 | PCYZ00000000 |
| Lactobacillus crispatus | Turkey | UMNPBX12 | MO-F2-W6-B4-C10 | PRJNA412075 | SAMN07702363 | PCYY00000000 |
| Lactobacillus aviarius | Turkey | UMNLAv97 | SNF2W6B3L1 | PRJNA316009 | SAMN04573117 | PCZP00000000 |
| Lactobacillus aviarius | Turkey | UMNLAv98 | SNF2W6B3L3 | PRJNA316009 | SAMN04573118 | PCYZ00000000 |
| Lactobacillus vaginalis | Turkey | UMNPBX13 | SNF2W6B1M2 | PRJNA412075 | SAMN07702364 | PCYX00000000 |
| Lactobacillus gallinarum | Turkey | UMNPBX14 | SNF2W6B4L1 | PRJNA412075 | SAMN07702365 | PCYW00000000 |
| Lactobacillus crispatus | Turkey | UMNPBX15 | SNF2W6B5L2 | PRJNA412075 | SAMN07702366 | PCYV00000000 |

TABLE 3D
Wild card combination

| | Host | | | | | |
|---|---|---|---|---|---|---|
| Target Strain | source | Strain name | Strain ID (internal) | Bioproject | Biosample | Genbank Accession |
| Lactobacillus crispatus | Turkey | UMNPBX16 | SNF2W6B1L3 | PRJNA412075 | SAMN07702367 | PCYU00000000 |
| Lactobacillus gallinarum | Turkey | UMNPBX17 | Lacto-spp-9-G9 | PRJNA412075 | SAMN07702368 | PCYT00000000 |
| Lactobacillus reuteri | Turkey | UMNPBX18 | SNF2W5B2M2 | PRJNA412075 | SAMN07702369 | PCYS00000000 |
| Lactobacillus ingluviei | Turkey | UMNPBX19 | MOF2W5B3T10 | PRJNA412075 | SAMN07702370 | PCYR00000000 |
| Pediococcus acidilactici | Turkey | UMNPBX20 | MOF2W5B3T3 | PRJNA412075 | SAMN07702371 | PCYQ00000000 |
| Lactobacillus johnsonii | Turkey | UMNLJ94 | SNF2W1B5L4 | PRJNA316010 | SAMN04573218 | PCZK00000000 |
| Lactobacillus johnsonii | Turkey | UMNLJ21 | MO-F2-W2-B3-C5 | PRJNA316010 | SAMN04573145 | CP021701, CP021702, |
| | | | | | | and CP021703 |
| Lactobacillus aviarius | Turkey | UMNLAv97 | SNF2W6B3L1 | PRJNA316009 | SAMN04573117 | PCZP00000000 |
| Lactobacillus aviarius | Turkey | UMNLAv98 | SNF2W6B3L3 | PRJNA316009 | SAMN04573118 | PCZO00000000 |
| Lactobacillus aviarius | Human | DSM 20655 | ATCC #1 | PRJNA222257 | SAMN02369392 | NZ_AYZA00000000.1 |
| Lactobacillus aviarius | Human | DSM 20653 | ATCC #2 | PRJNA222257 | SAMN02369423 | NZ_AYYZ00000000.1 |
| Clostridium bartletii | Human | DSM 16795 | ATCC Strain | PRJNA245632 | SAMN02745147 | FUXV00000000.1 |

### Testing and Results

The 4-strain probiotic blend, 10-strain (3 week) probiotic blend, and 10-strain (6 week) probiotic blend (described above) were tested for their ability to enhance turkey performance in two separate trials.

In the first trial, day-of-hatch turkey poults in a caged trial with 5 replicate cages per treatment group (n=50 per treatment) were inoculated via oral gavage with either a negative saline control (Negative control), a commercially available probiotic derived from chicken isolates (FM-B11, applied according to manufacturer's instructions), or 1×10⁸ CFU of each of the strains described above for the 4-strain combination and the two 10-strain combinations. Birds were weighed at four time points. Feeds were weighed back at each sampling time point to calculate feed conversion rate. In this study, all of the novel turkey-source strain combination groups had significantly higher final body weights than the saline control and the commercial chicken source probiotic (P<0.05).

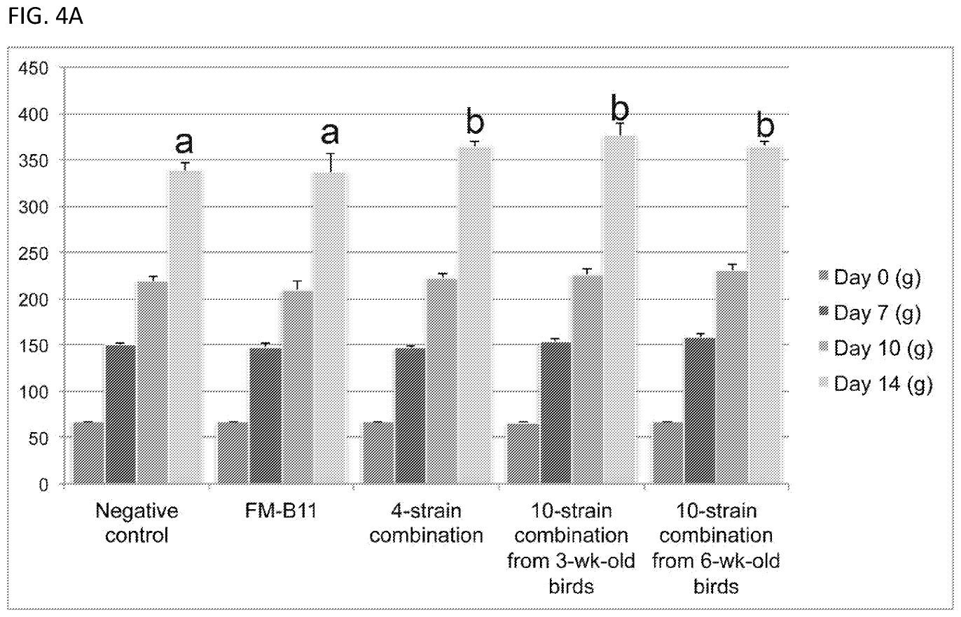

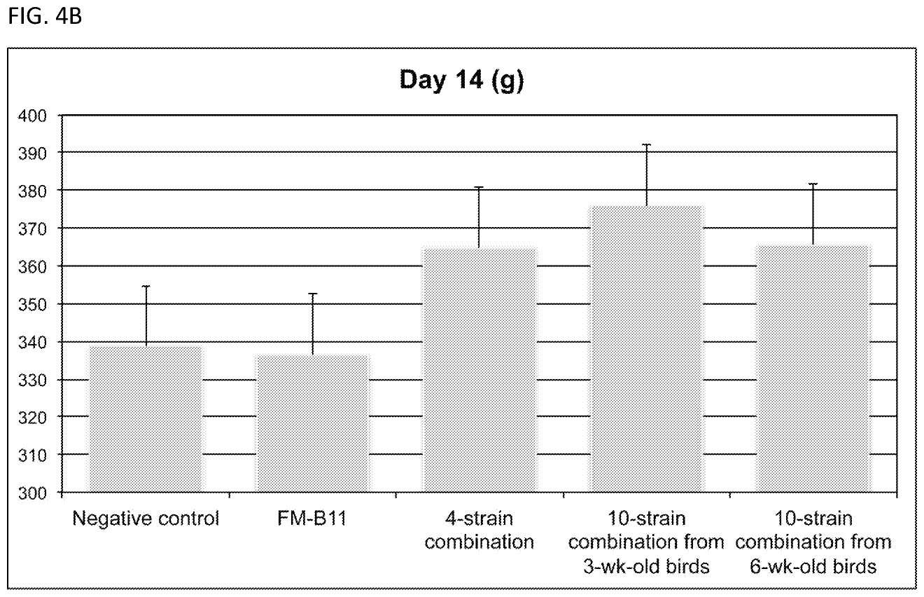

Results are shown in FIG. 4 and Table 4. These results indicate that the strain combinations performed equally well at enhancing turkey poult body weights over the course of 14 days in cages.

TABLE 4
Average body weights for turkey poults administered various
probiotic combinations. SEM = standard error of means.

| | Day 0 | Day 0 | Day 7 | Day 7 | Day 10 | Day 10 | Day 14 | Day 14 |
|---|---|---|---|---|---|---|---|---|
| | (g) | SEM | (g) | SEM | (g) | SEM | (g) | SEM |
| Negative control | 66.1 | 0.3 | 149.7 | 4.4 | 219.5 | 7.9 | 338.6 | 16.1 |
| FM-B11 | 66.1 | 0.3 | 147.7 | 4.4 | 209.7 | 7.9 | 336.5 | 16.1 |
| 4-strain combination | 66.3 | 0.3 | 146.5 | 4.4 | 223 | 7.9 | 364.8 | 16.1 |
| 10-strain combination | 66 | 0.3 | 153 | 4.4 | 226.5 | 7.9 | 375.9 | 16.1 |
| from 3-wk-old birds | | | | | | | | |
| 10-strain combination | 66.6 | 0.3 | 157.8 | 4.4 | 230.2 | 7.9 | 365.6 | 16.1 |
| from 6-wk-old birds | | | | | | | | |

Feed conversion was also assessed over the course of the experiment. Average feed conversion rate was reduced in all treatment groups from 1.35 (Control) to 1.29 (FM-B11), 1.31 (4-strain combination), 1.29 (10-strain combination from 3-wk-old birds), and 1.32 (10-strain combination from 6-wk-old birds). All groups except the 10-strain combination from 6-wk-old birds were significantly different from the negative control group.

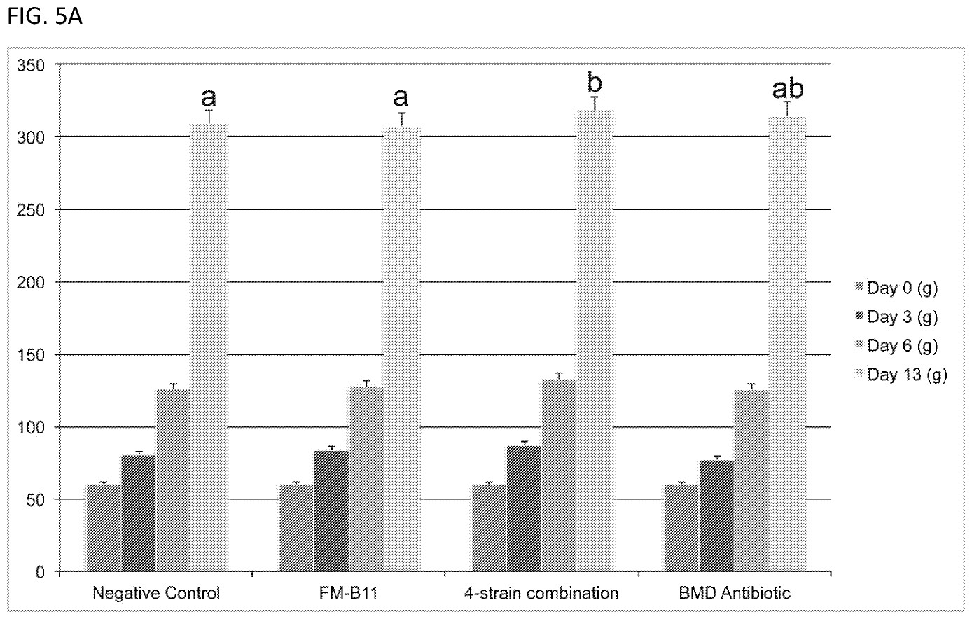

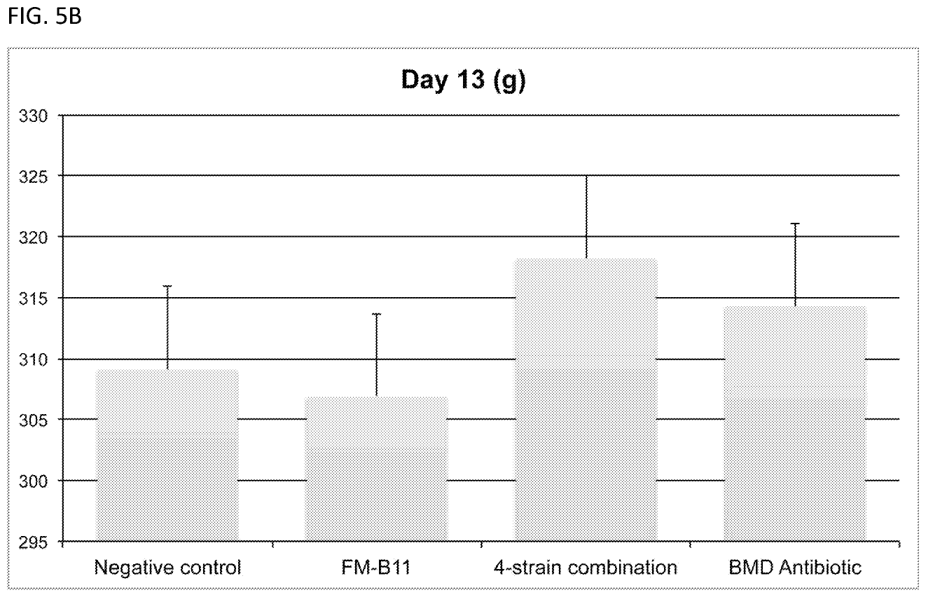

A second trial was performed to repeat the 4-strain combination inoculation and compare it again with a negative saline control, an existing commercial probiotic derived from chickens (FM-B11), and a low-dose antibiotic (bacitracin methylene disalicylate or BMD) administered continuously at 50 g/ton in the feed. In this trial, the 4-strain combination again displayed significantly higher final body weights than the saline control and the commercial FM-B 11 probiotic (P<0.05). Results are shown in FIG. 5 and Table 5 and indicate that there is repeatability in the ability of this 4-strain combination to enhance turkey performance related to weight gain.

TABLE 5
Average body weights for turkey poults administered various
probiotic combinations. SEM = standard error of means.

| | Day 0 | Day 0 | Day 3 | Day 3 | Day 6 | Day 6 | Day 13 | Day 13 |
|---|---|---|---|---|---|---|---|---|
| | (g) | SEM | (g) | SEM | (g) | SEM | (g) | SEM |
| Negative control | 60.1 | — | 80.7 | 1.3 | 125.7 | 2.4 | 309.1 | 6.8 |
| FM-B11 | 60.1 | — | 83.5 | 1.3 | 127.8 | 2.4 | 306.9 | 6.8 |
| 4-strain combination | 60.1 | — | 86.9 | 1.3 | 132.8 | 2.4 | 318.2 | 6.8 |
| BMD Antibiotic | 60.1 | — | 76.8 | 1.3 | 125.5 | 2.4 | 314.3 | 6.8 |

### Example 3

Pen trial experiments were performed using the 4-strain probiotic blend and 10-strain (3 week) probiotic blend described in Example 2. The effect of each combination was assessed alone and in combination with one of two prebiotics, Lactose 1% in feed or 0.5 lbs SAFMANNAN per ton of feed (Phileo Lesaffre Animal Care, Marcq-En-Baroeul Cedex, France). The prebiotic was administered continuously in feed. The experimental design is shown in Table 6.

TABLE 6
Experimental design for pen trials.

| Group | Pens per group | Birds per group |
|---|---|---|
| Control | 7 | 168 |
| SAFMANNAN | 7 | 168 |
| Lactose 1% | 7 | 168 |
| 4-strain probiotic blend | 7 | 168 |
| 10-strain probiotic blend | 7 | 168 |
| SAFMANNAN + 4-strain blend | 7 | 168 |
| SAFMANNAN + 10-strain blend | 7 | 168 |
| Lactose 1% + 4-strain blend | 7 | 168 |
| Lactose 1% + 10-strain blend | 7 | 168 |
| Total | 63 | 1512 |

This Experiment started in December 2017 and lasted 15 weeks. Poults were housed in pens through 6 weeks of age then moved to a common area. They remained on experimental feed through 6 weeks; then were all given control feed. Probiotic inoculations were performed at day of hatch and 3 weeks of age.

Prior to shared housing, the combination of prebiotic with probiotic consistently resulted in the most enhanced bird weights, as compared to the control group or prebiotic or probiotic alone. Significant body weight enhancements in birds treated with the combination of prebiotic with probiotic compared to control birds and, in some cases, in birds treated with probiotic alone were observed at weeks 2 and 3 of age, and body weight trended higher than controls throughout the experiment (Table 7). The best enhancements in body weights from control ranged from 2% to 6% across these time points.

TABLE 7
Body weights of turkey poults fed different
combinations of prebiotics and probiotics.

| | Total body weight average (grams) | | | | |
|---|---|---|---|---|---|
| Treatment | Day 0 | Week 1 | Week 2 | Week 3 | Week 6 |
| Control | 64.2 | 171.5 | 343.0 | 693.0 | 2420.0 |
| SAFMANNAN | 64.6 | 172.0 | 352.1 | 712.0 | 2424.0 |
| Lactose 1% | 65.0 | 169.9 | 350.7 | 704.0 | 2471.0 |
| 4-strain probiotic blend | 64.8 | 168.7 | 347.6 | 709.0 | 2440.0 |
| 10-strain probiotic blend | 65.0 | 167.9 | 342.6 | 704.0 | 2463.0 |
| SAFMANNAN + | 65.0 | 177.0 | 359.3 | 730.0 | 2473.0 |
| 4-strain blend | | | | | |
| SAFMANNAN + | 64.8 | 173.2 | 357.1 | 723.0 | 2474.0 |
| 10-strain blend | | | | | |
| Lactose 1% + | 65.0 | 174.0 | 361.7 | 728.0 | 2469.0 |
| 4-strain blend | | | | | |
| Lactose 1% + | 64.9 | 175.4 | 352.6 | 712.0 | 2476.0 |
| 10-strain blend | | | | | |
| Standard error of means | 0.4 | 2.3 | 4.0 | 10.0 | 20.0 |
| P value Diet | 0.54 | 0.27 | 0.01 | 0.04 | 0.52 |
| P value Probiotic | 0.84 | 0.53 | 0.09 | 0.03 | 0.33 |

A significant positive effect on bird uniformity was observed with treatment at week 6 of age and probiotic administration at week 3 of age (Table 8). These enhancements trended throughout the experiment, particularly with prebiotic+probiotic administration. Feed conversion was significantly enhanced from 0-2 weeks of age, with a 0.03-0.10 enhancement with prebiotic+probiotic treatment groups compared to control (Table 9). This trend was not observed across the entire experiment.

Overall, this experiment demonstrated that the probiotic blends tested had the ability to enhance performance in pen trials mimicking real life conditions, resulting in higher body weights, improved feed conversion, and increased bird uniformity. The addition of prebiotics further enhanced these effects of the probiotic blends.

TABLE 8
Coefficient of variation as a measure of
bird uniformity at weeks 3 and 6 of age.

Coefficient of variation

| Treatment | Week 3 | Week 6 |
|---|---|---|
| Control | 10.426 | 9.752 |
| SAFMANNAN | 11.696 | 8.5166 |
| Lactose 1% | 10.304 | 9.3181 |
| 4-strain probiotic blend | 9.5075 | 9.9281 |
| 10-strain probiotic blend | 9.6519 | 9.4035 |
| SAFMANNAN + 4-strain blend | 8.4479 | 7.9967 |
| SAFMANNAN + 10-strain blend | 9.1229 | 8.1494 |
| Lactose 1% + 4-strain blend | 9.2001 | 9.0704 |
| Lactose 1% + 10-strain blend | 8.8312 | 8.8141 |
| Standard error of means | 0.63 | 0.67 |
| P value Diet | 0.81 | 0.06 |
| P value Probiotic | 0.02 | 0.97 |

TABLE 9
Feed conversion ratios from 0-2 and 0-6 weeks of age.

Feed conversion ratio

| Treatment | 0-2 weeks | 0-6 weeks |
|---|---|---|
| Control | 1.43 | 1.71 |
| SAFMANNAN | 1.38 | 1.74 |
| Lactose 1% | 1.40 | 1.71 |
| 4-strain probiotic blend | 1.42 | 1.72 |
| 10-strain probiotic blend | 1.43 | 1.71 |
| SAFMANNAN + 4-strain blend | 1.36 | 1.72 |
| SAFMANNAN + 10-strain blend | 1.40 | 1.72 |
| Lactose 1% + 4-strain blend | 1.34 | 1.71 |
| Lactose 1% + 10-strain blend | 1.40 | 1.71 |
| Standard error of means | 0.01 | 0.01 |
| P value Diet | 0.00 | 0.37 |
| P value Probiotic | 0.01 | 0.87 |

The complete disclosure of all patents, patent applications, and publications, and electronically available material (including, for instance, nucleotide sequence submissions in, for example, GenBank and RefSeq, and amino acid sequence submissions in, for example, SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A composition comprising at least 4 bacterial species or strains selected from the following bacterial species:
Clostridium bartlettii;
Lactobacillus acidophilus;
Lactobacillus aviarius;
Lactobacillus crispatus;
Lactobacillus gallinarum;
Lactobacillus helveticus;
Lactobacillus ingluviei;
Lactobacillus johnsonii;
Lactobacillus reuteri;
Lactobacillus salivarius;
Lactobacillus vaginalis; and
Pediococcus acidolactici;
wherein at least one of the bacterial species or strains is an avian-sourced strain.

2. (canceled)

3. The composition of claim 1, wherein at least one of the bacterial species or strains is a turkey-sourced strain.

4. The composition of claim 1, wherein the bacterial species or strains comprise
Clostridium bartlettii strain DSM 16795 (deposited in the NCBI database under GENBANK accession number FUXV00000000.1);
Lactobacillus acidophilus strain UMNPBX11 (deposited in the NCBI database under GENBANK accession number PCYZ00000000);
Lactobacillus helveticus strain UMNPBX6 (deposited in the NCBI database under GENBANK accession number PCZD00000000); or
Lactobacillus ingluviei strain UMNPBX19 (deposited in the NCBI database under GENBANK accession number PCYR00000000); or
a combination thereof.

5. (canceled)

6. The composition of claim 1, wherein the bacterial species or strains comprise Lactobacillus aviarius subspecies araffinosus or Lactobacillus aviarius subspecies aviarius or both.

7. The composition of claim 1, wherein the bacterial species or strains comprise:
Lactobacillus aviarius strain UMNLav12 (deposited in the NCBI database under GENBANK accession number NZ_LWUE00000000.1);
Lactobacillus aviarius strain UMNLav13 deposited in the NCBI database under GENBANK accession number NZ_LWUF00000000.1);
Lactobacillus aviarius strain UMNLAv76 (deposited in the NCBI database under GENBANK accession number PCZQ00000000);
Lactobacillus aviarius strain UMNLav97 (deposited in the NCBI database under GENBANK accession number PCZP00000000);
Lactobacillus aviarius strain UMNLav98 (deposited in the NCBI database under GENBANK accession number PCZO00000000);
Lactobacillus aviarius strain DSM 20653 (deposited in the NCBI database under GENBANK accession number NZ_AYYZ00000000.1); or
Lactobacillus aviarius strain DSM 20655 (deposited in the NCBI database under GENBANK accession number NZ_AYZA00000000.1); or
a combination thereof.

8. The composition of claim 1, wherein the bacterial species or strains comprise:
Lactobacillus crispatus strain UMNPBX1 (deposited in the NCBI database under GENBANK accession number PCZI00000000);
Lactobacillus crispatus strain UMNPBX8 (deposited in the NCBI database under GENBANK accession number PCZB00000000);
Lactobacillus crispatus strain UMNPBX12 (deposited in the NCBI database under GENBANK accession number PCYY00000000);
Lactobacillus crispatus strain UMNPBX15 (deposited in the NCBI database under GENBANK accession number PCYV00000000); or
Lactobacillus crispatus strain UMNPBX16 (deposited in the NCBI database under GENBANK accession number PCYU00000000); or
a combination thereof.

9. The composition of claim 1, wherein the bacterial species or strains comprise:
Lactobacillus gallinarum strain UMNPBX4 (deposited in the NCBI database under GENBANK accession number PCZF00000000);
Lactobacillus gallinarum strain UMNPBX14 (deposited in the NCBI database under GENBANK accession number PCYW00000000); or
Lactobacillus gallinarum strain UMNPBX17 (deposited in NCBI databases under GENBANK accession number PCYT00000000); or
a combination thereof.

10.-11. (canceled)

12. The composition of claim 1, wherein the bacterial species or strains comprise of:
Lactobacillus johnsonii strain UMNLJ21 (deposited in the NCBI database under GENBANK accession numbers CP021701, CP021702, and CP021703);
Lactobacillus johnsonii strain UMNLJ94 (deposited in the NCBI database under GENBANK accession number PCZK00000000); or
Lactobacillus johnsonii strain UMNLJ113 (deposited in the NCBI database under GENBANK accession number PCZJ00000000); or
a combination thereof.

13. The composition of claim 1, wherein the bacterial species or strains comprise:
Lactobacillus reuteri strain UMNPBX3 (deposited in the NCBI database under GENBANK accession number PCZG00000000);
Lactobacillus reuteri strain UMNPBX7 (deposited in the NCBI database under GENBANK accession number PCZC00000000);
Lactobacillus reuteri strain UMNPBX10 (deposited in the NCBI database under GENBANK accession number PCZL00000000); or
Lactobacillus reuteri strain UMNPBX18 (deposited in the NCBI database under GENBANK accession number PCYS00000000); or
a combination thereof.

14. The composition of claim 1, wherein the bacterial species or strains comprise:
Lactobacillus salivarius strain UMNPBX2 (deposited in the NCBI database under GENBANK accession number PCZH00000000); or
Lactobacillus salivarius strain UMNPBX9 (deposited in the NCBI database under GENBANK accession number PCZA00000000); or
a combination thereof.

15. The composition of claim 1, wherein the bacterial species or strains comprise:
Lactobacillus vaginalis strain UMNPBX5 (deposited in the NCBI database GENBANK accession number PCZE00000000); or
Lactobacillus vaginalis strain UMNPBX13 (deposited in the NCBI database under GENBANK accession number PCYX00000000); or
a combination thereof.

16. The composition of claim 1, wherein the bacterial species or strains comprise Pediococcus acidolactici UMNPBX20 (deposited in the NCBI database under GENBANK accession number PCYQ00000000).

17. The composition of claim 1, the composition comprising at least 4 bacterial species or strains selected from the following:
Clostridium bartlettii;
Lactobacillus aviarius; and
Lactobacillus johnsonii.

18. The composition of claim 17, wherein the bacterial species or strains comprise:
Clostridium bartlettii DSM 16795 (deposited in the NCBI database under accession number FUXV00000000.1);
Lactobacillus aviarius strain UMNLav12 (deposited in the NCBI database under accession number NZ_LWUE00000000.1);
Lactobacillus aviarius strain UMNLav13 (deposited in the NCBI database under accession number NZ_LWUF00000000.1); and
Lactobacillus johnsonii strain UMNLJ21 (deposited in the NCBI database under accession numbers CP021701, CP021702, and CP021703).

19. The composition of claim 1, the composition comprising at least 4 bacterial species or strains selected from the following:
Lactobacillus aviarius;
Lactobacillus crispatus;
Lactobacillus gallinarum;
Lactobacillus helveticus;
Lactobacillus johnsonii;
Lactobacillus reuteri;
Lactobacillus salivarius; and
Lactobacillus vaginalis.

20. (canceled)

21. The composition of claim 1, the composition comprising at least 4 bacterial species or strains selected from the following:
Lactobacillus acidophilus;
Lactobacillus aviarius;
Lactobacillus crispatus;
Lactobacillus gallinarum;
Lactobacillus johnsonii;
Lactobacillus reuteri;
Lactobacillus salivarius; and
Lactobacillus vaginalis.

22. (canceled)

23. The composition of claim 1, the composition comprising at least 4 bacterial species or strains selected from the following:
Clostridium bartlettii;
Lactobacillus aviarius;
Lactobacillus crispatus;
Lactobacillus gallinarum;
Lactobacillus ingluviei;
Lactobacillus johnsonii;
Lactobacillus reuteri; and
Pediococcus acidolactici.

24.-34. (canceled)

35. A composition comprising at least one bacterial strains selected from the following:
Clostridium bartlettii strain DSM 16795 (deposited in the NCBI database under GENBANK accession number FUXV00000000.1);
Lactobacillus acidophilus strain UMNPBX11 (deposited in the NCBI database under GENBANK accession number PCYZ00000000);
Lactobacillus helveticus strain UMNPBX6 (deposited in the NCBI database under GENBANK accession number PCZD00000000);
Lactobacillus ingluviei strain UMNPBX19 (deposited in the NCBI database under GENBANK accession number PCYR00000000);
Lactobacillus aviarius strain UMNLav12 (deposited in the NCBI database under GENBANK accession number NZ_LWUE00000000.1);
Lactobacillus aviarius strain UMNLav13 deposited in the NCBI database under GENBANK accession number NZ_LWUF00000000.1);
Lactobacillus aviarius strain UMNLAv76 (deposited in the NCBI database under GENBANK accession number PCZQ00000000);
Lactobacillus aviarius strain UMNLAv97 (deposited the NCBI database under GENBANK accession number PCZP00000000);
Lactobacillus aviarius strain UMNLAv98 (deposited in the NCBI database under GENBANK accession number PCZO00000000);
Lactobacillus aviarius strain DSM 20653 (deposited in the NCBI database under GENBANK accession number NZ_AYYZ00000000.1);
Lactobacillus aviarius strain DSM 20655 (deposited in the NCBI database under GENBANK accession number NZ_AYZA00000000.1);
Lactobacillus crispatus strain UMNPBX1 (deposited in the NCBI database under GENBANK accession number PCZI00000000);
Lactobacillus crispatus strain UMNPBX8 (deposited in the NCBI database under GENBANK accession number PCZB00000000);
Lactobacillus crispatus strain UMNPBX12 (deposited in the NCBI database under GENBANK accession number PCYY00000000);
Lactobacillus crispatus strain UMNPBX15 (deposited in the NCBI database under GENBANK accession number PCYV00000000);
Lactobacillus crispatus strain UMNPBX16 (deposited in the NCBI database under GENBANK accession number PCYU00000000);
Lactobacillus gallinarum strain UMNPBX4 (deposited in the NCBI database under GENBANK accession number PCZF00000000);
Lactobacillus gallinarum strain UMNPBX14 (deposited in the NCBI database under GENBANK accession number PCYW00000000);
Lactobacillus gallinarum strain UMNPBX17 (deposited in NCBI databases under GENBANK accession number PCYT00000000);
Lactobacillus johnsonii strain UMNLJ21 (deposited in the NCBI database under GENBANK accession numbers CP021701, CP021702, and CP021703);
Lactobacillus johnsonii strain UMNLJ94 (deposited in the NCBI database under GENBANK accession number PCZK00000000);
Lactobacillus johnsonii strain UMNLJ113 (deposited in the NCBI database under GENBANK accession number PCZJ00000000);
Lactobacillus reuteri strain UMNPBX3 (deposited in the NCBI database under GENBANK accession number PCZG00000000);
Lactobacillus reuteri strain UMNPBX7 (deposited in the NCBI database under GENBANK accession number PCZC00000000);
Lactobacillus reuteri strain UMNPBX10 (deposited in the NCBI database under GENBANK accession number PCZL00000000);
Lactobacillus reuteri strain UMNPBX18 (deposited in the NCBI database under GENBANK accession number PCYS00000000);
Lactobacillus salivarius strain UMNPBX2 (deposited in the NCBI database under GENBANK accession number PCZH00000000);
Lactobacillus salivarius strain UMNPBX9 (deposited in the NCBI database under GENBANK accession number PCZA00000000);
Lactobacillus vaginalis strain UMNPBX5 (deposited in the NCBI database GENBANK accession number PCZE00000000);
Lactobacillus vaginalis strain UMNPBX13 (deposited in the NCBI database under GENBANK accession number PCYX00000000); or
Pediococcus acidolactici strain UMNPBX20 (deposited in the NCBI database under GENBANK accession number PCYQ00000000).

36. The composition of claim 35 comprising at least two of the listed bacterial strains.

37. The composition of claim 36 comprising at least three of the listed bacterial strains.


SEQUENCE LISTING (49 sequences)

SEQ ID NO: 1 - DNA, 1515 bp, L. johnsonii
ttgaagaagaaagttgctggtgaagttcaaacagctaagggcaatattgatgctgccactaaagacgctgatgttaagacagcgcaaactaatggtgaaaaagctattaatgctgttgaaatccctgcttcagtttctaagactgatgccattaaagcaatcaatgctgcattaacagataagaaggctaaacttaacggtactaacttaactgatgaagaaaaaaccgcagtaattaacaaagcacaaaagcttgctgatgacgcaattgctaacattaataaagcaacaactaacgatgcagtaaacactgctaaggaaaatggtgttcaagctattaagaacatgacaattcctacagtttcagatgttaagaagaacgctaaggatgccattgataaagctgctaagactaaggacaatgcaatcgatgcttcaaacttaactactgaagaaaaggatgctttgaagaagaaagttgctggtgaagttaaaatagctaaggacaatattgatgctgccactaaagacgctgatgttactacagcgcagaccaatggtaaaaaggctattaaagcagttgaaattccaacatcttctaagaccaagaatgacgctaatagtgatctcgataacacagctgctgctgttaagcaggctatcgataaaacttcaggtttaactgatgatcaaaagaaaacagcaaaggatcaaattgaccaagctgttgatgatgctaaagaaaacatcaagaatgcttctgataacaaaggtattgcagatgcaacggatgcaggtaagctcgcaatcgacaaggtaagtgcaaaggctgcaattgatgcagcagttgccgctaagaagagcaaaattgcgaaggcgccattaacttctgaagaagccaagcttttaaacaacttggtagatcaagaagctgacgctgcaaaggacgctattgatgccgctactactaatgcggttgttgaagctgccaagaataatggtgttgaaacgattaacaacattaatgttcctgctacttccgcaactaaagatgataccaacaaggcaattgatgatgctttagctaaaaagattgaagaaatcaacaatgctaacttaactgatgaccaaaaacaaagccttattgatcaagctcagaacgcagctaaccaggctaaggaaaacgttcggagtgcttccactgatgaagacgttcaaactgctaagaacaatggaattgcagctattaacggaattactgttaagagtaattcagtcgatgaccaagataattctgcaactaatgaagggaacggtaaccaagctggtcatattcaaagtgataactccagtgacgttacaaagcatagttctactcaacagtctggtaatgaaaaagataaattaccacaaactggtaacgaaatacaacgtggtgcaggcttggttggcttagccattgcaggcttagctattttgttgggcagtgccggattccgtaaaaagcgggattaa

SEQ ID NO: 2 - DNA, 1158 bp, L. johnsonii
ttgtatattcgaactatccttgcttcgattacatcagaagaaaaaataaattcttttgatccattgattcatacaggaaatataaaaaaagattatttagataattttgatttatttactatagttagatatttttccagctcaagtgatgtatataaattatttgaaaggtatcaatcgctatcttttattgatttagataacgaaggaattgaatatttagaaaaaattattccaaatattctttcttcacattacaaagatgaattgaggaatgatatctattggcgggtattatgtgtttgtagctttgtaaaattaaataaaaatatagtaaatctaataattgaaaactttactgactatttagttgatttagatactattgagcatagagacattattagacagttttttataaatataaaaaatcagaatttagttaataatcaaaatattagtcaaatacaaaaaatcgcaactcaagtcatgatgatagttatcagaagtaaggcagaaaacatatcctcaataatttatttttttaaccattatttgaacgaagaagatactgagtatgattataataaaatagttgacgaagctatgaagaataattatgacattgctcttatttatacttatgagttttcaaacaaggggacacaggatctaataacaacttactttaaaaatagatcatttcaaagtaaagagattgaaataaaaacaaacatagatcttgcaaactttgacattattgagttagatgaaaagattgagaatgatatacttaattacttaaaaacaaaggatactcgtgcagaagatgaacatatgctttctcaggatattcgacaaataactcatggtttcactaaagtatgtatagagaagaaagaaaagatagataaggacacgatagatgttattagcaaatatgctatgccggaagatagatggttaatcaattataaaaatttcaattacgaaaagtttaaactagattggttaaaaagatgtgatcgtaatattttgaaacaaatttctgcaaataaggttgttaagaatgagataaaaaatagattaattcaaagttttaaaaacaataaattatcgtctgaattggaagaaatctatttttattatttcgcataa

SEQ ID NO: 3 - DNA, 2781 bp, L. johnsonii
atggacgtaacaacacaaaaaagaaatgcaaaacaatttatacaagattggaaagatcgtggaagagaaaaacaagacagtcaatcattttggctgtctttgttgcgtgaagttttaggtgttgaaaatccagaaaacttcattaagtttgaagaaaaagtaaaattatcacacgatagttttattgatggctacattgagcaaacacatgtgatgattgaacaaaaaggaagtaataaggatttagataaggcaattaaacaatcagatgggaggttattaacaccctttcaacaggctcaaagatattctgctgcattaccatattcacgccgaccacgttggattgttacttgtaactttagagaatttcgtatctatgatatggaacatcctaattcagaaccggtgaagattgaattaaaagaccttgaaactagctattatcaacttgaattcttagtagataagagtaatgaacatctagaaaaagaaaaacaggtttccttaagtgcgggtgaactggttggtcaaatttatgatgaacttttagatcaatacaaaaatccagaaaatgagcattcacaaaaaagtattaatcaactctgtgtcagaattgtattttgtctttatgcagaagatgctggcatctttggtaagaaaaatatgtttcatgattaccttgaagaatttgattctagacatataagaggtgctttgattaggttattcaaggtattagatactaaagttaaagaccgtgatccttatttagctgatgatgatccaaaattagcagaatttccttatgttaatggtggaatgttttcggatgaaaatattgaaattccaccttttactgatgaattgagagatctactccttcgtaaagccagtgatgaatttgattggtctgagattagtcctactatttttggcgcggtatttgagtcaacattaaaccctgaaacaagacgtcagggagggatgcactatacttctgttgaaaatattcataaagtaattgatccattattcatggctggtttaaaaaatgaattaaatgaaattaaaaagactaaacaacctgctatattaaaaaagaaggctagtgcatttcaagaaaaattagcaaacctaacattctttgatcctgcatgtggttcaggcaatttcttaacagaaacatatttgcaacttagacgcttggaaaatgatgctattaaattgatttatccgaatccttcacttgatgttggtcaggctaaagatattattaaagtctctattaagcaattttatggaattgaaattaatgactttgctgtatctgttgccaagaccgctttatggattgcggagagccagatgctagaagaaactaaagatattttttatgccgattgggatttcttacctttaaaaacatatacgaatattcatgaaggcaatgctttaaggatggattggaatagggtagttcctaactatgcttgtcattatattatgggcaatccgccctttatcggacatcaatggagaaatagtgagcaaataagagatatggaaatagtttttcatgatttaggcaaacatggaaaattagattatgtttgtgcctggtataataaggcgataaattatatgcataaaactaagattagggctgccttggtttctacgaattctatttgtcaaggagaatcagtttctattctttggcaatacttggaaaaaaagaatctagaaattcagtttgcttataacacctttgtatggacaagtgaagctaagagcaaagcggcagtacactgtgtaataattggttttacagattataaaactaaagatgataaattcatatttgacgatggtaaaattaagaaagtaaagcatataaatggctatttattggatgcaccagatgtttttatacaatccagaggaaaaataagatttggaaattttcctaagatgactaagggaagccagcctacagatggtaataacctaattttgaatgagcaggaatatcaacaatttattagagaatatccagatagtatagacttgattcgtccatatatgggaagtagagaatttatcaataatcagcaaagatattgtttatggttagtaaataccaatccaaaagacattaggaacaacaaattcatccttgaaagaatagatagggttaaatcatttagaaaaaatagtaaaactgcttcagtgagagaagatgcctctacaccaactttatttacgcaaatcagacaacctgatactaactatttagttgttccagcaatgtcaagtggtaaaagaagatatgttccaattggatttattaaaaaagatattatcgcaagcaatcagttatatataattcctacagattcaatatatttgtttggtttgttaacatctaatatgcacatggcgtggatgagaacggtatgtggtagattgaaaagcgattataggtatgcaccagcaatttataataacttcccatggcctaagtcaactaaagaacaaaaagctaaaatagaagagacagcaaaagctatcttagacgcacgtgcattatatccagatagttctctagctgatctctatgatccgttaactatgccaagagaattgcttaaggcgcatcaaaataatgatcgtgcagtgatggaagcctatgggttacctgttaagggaactacagaaagtgatgctgttgctcacttgtttaagatgtatgaaaaattgatagctggggaaaagtga

SEQ ID NO: 4 - DNA, 1995 bp, L. johnsonii
atgagcttaaattatatagacttattttctggagctggcggccttggtctaggttttgaaagagccggtttcaagaatatattctctgttgaatttaataaagaaatttcagagacgtataagtataatttcccaaataatcaccttattgttgatgatataaaaaatataacagcagcacaaataaaaaatttaaaaggagatagtgatgtagatgtaataattggaggtcccccatgtcaaggcttcagtatggctggaaatattggtagaaaattcgtagatgatccaagaaaccatttatttagagaatttgttcgggttgttgatattgtaaaacccagaatgtttgtcatggagaatgttgctagaatggcaacacataatcatgggaaaactattgaagaagtatgtaatgaattcgagaaaattgggtatcatgtacaatataaagttttaaattcagtctactatagtgttccccagaatagacgtagaatatttgttgtgggagtttctgataatatggattttgggtatcctaccccatcatttgacttaaaaacggtaaaggatgctattgacgatttgccaaaacttggaaatggggaaagatcagatgttccaaatcattttgctatgaaacatacacagcaaatgcttgataaaatggcatatgttaaagaaggaggcagtaaacaagatattccggaaagcattagacctaaatctggtgatgctcgcaaatatattagatatgatcggaataaaccttcagtaacaatcacaggagatatgcgtaaagtatttcattataatcagaaccgtgcattaactccacgagaactagcacgtatacaaacattccctgataattttatttttcaaggaaatagtatatccattcaacagcaaattggtaatgctgttcctcctcttctggctaaagcaatggctaactctgtaaaacaagctttaaccataatttcaaatcataaatatcctaaagttaattacataggaaataaggaaaaattatcaacatggattgcagatagtatgcccgataaaactaaatcagttttagatctttttgctgggggtaattctatttcttatgaattaaagaaacgaggatataaagttatatctaatgatgcacttttttcttcatttgctctgtcaaaaggattaatagaaaataaaaatactatattagatgaagaggtagtaaaacatgctcttacagtgcctgtagatactactgatctaaataaggttaattggcttgcagataatttatatttttcaaatgaaatggaagaattagccaaattggtaaaatattcttattctcttgaaggatatcaacgatatctttatttatctctcttaagaagagcgatgattcgcaaattaccatactcaaggatgaatttagattggaaaaatataaaaaaattaagagatgaagattattcttatcgaaagtataaaagacgccgtgcatatcataatcaaactttcgcgcatcatatgttagctgagcttgaatcatataataaagcgatttttgataatggagaaaataatattgctcttcatttagatgcagtatcggctttaaattctattgattcagttgatgtaatttatatggatcccccgtatccaggaacaatgaataattatgatggattttatgggaaatatgataaagtattcaacgaacacataaaatttgatgatttaacacaaagctctacgtttcttaaagaactagagcatttgattgaattatcttcaatcaagtcaaaatacttatttttaagtattaattcaaggataaaaccttcatatgaagatgtattaaatttatgttcgtactatggcgacgtcactttgcataaaaaaaagcataattatcaagtaagtggtaaggacaacaaaaataaaaatatggagttacttattgaagtaaaattttataaataa

SEQ ID NO: 5 - DNA, 1737 bp, L. johnsonii
atgaacacaaaagaaatagaagtattaagaacaataaaaccaggtattagccaaagagatttagctaaaaagcttaatatctcacttggaaaagttaatcagataattagtgaattaaaaaggcaaagatttatagaccaaaataacaatattactactaaggccgaaatatattttgagtctcatcatccaaaaaatgcaattattcttgcagcaggatatggaatgagaatggtgcctattaatactgaggaaccaaaaggattgttggaagttaaaggtgaaactttaattgaacgcttgattaaacaattacatgaagtgggaattaaaaatatcgaaattgtagtcggctttatgaaggaacattatgagtttttaattgataaatatcatgtcaatttagttgttaattctcattataaggattggaataatatctactctttatttttagttaataatgtcatatctaatacttatgttctcccttgtgatgtatggtttaaaactaacccattttctaccgttgaagatgaatcatggtatttatttggggaagaaatagtaacgggttcggattggcaagttaaaaataatggtaaggtgcgttttaaagcatcaaatggaaataaaatgattggtcttgcatatataaatagtacgcatggaaaaattgtttcaaaaagactagaagaaatgattaaaaaggagcgtcatacttctttctgggaagatgttttagaggataaaaagacgttcttgctcaatggtaaagttatttctgatgattcacatgctgaaattaattcttatgaacaattgctagatttagattcaggatcaacgcatttaaaaaataacgcaattgagattatcgaacaagttttaaaggtaaataaaaaagatattcataatattcatactttaaaaaaaggaatgacaaatagatctttcatttttacagttaataataaaagatatattatgcggattcctggtgaaggaacagataagctaatcgatcgaaaagaagagtatgatgtttaccagaaagtgaaaacagagccatatactgaaaaaatattatatttaaatccagataatggatacaaaatatctgaatttctagaaaatactagaaatagtgatgcaaataattttcaagatgttaaaaatagtatgagtgtcttacgacaatttcataagcaaaactatcaagttaatcatacttttgacatttggaggcagatagatctttatgaaagtcttagaaatacagattcagcttatcgagattattcagaaacaaagaacaatgttcttaagctaaaaaaatttattgaagataatattgatagatggtctttatgtcatatagatgccaattcagataacttcttgattgatcagaacgaaaatgtatttcttatcgattgggaatatgctggcatgcaggatccagatttggatattgcaatgtatgccatttatgctggctataataaagacaaaattgatgaactaatagatatttactatgaaaataaatgcaaagataatattagacataaaatttatgcttatgttgctgtaggaggactactgtggagtaactggtgtgaatataagcaatcattaggactagattttggcgaatattcaatcgcacaatatcgttatgccaaagagtacagtaaattagttttaaagtatttggagaagaagaatgactaa

SEQ ID NO: 6 - DNA, 1674 bp, L. johnsonii
atggttaaagaaattgatataaaaaaatataagaaatttgcagataagacgattaaatttgaaaaaagtttaaacgcaatttctggaaccaatggaactggcaaatcctcgcttttatatattattagtaatgcgtataagcgtgttaataaaagatgtgcttggataaaagatgaaaattgtttaagtattattaatagtattaactctgaagtaaatcctaaagtagaatcattaacacgtgagagtatggattatgtagatccagctcaaggtattaaaggtaatttatttaaggttaaatattatgatcataatgaattggaatttagaagacataattctcctaaaaattctagatatgctataaaaccaatatataaaaataattctcatgataaattaccacagtgtcctattatatatttaggactatctcgattaattccatatggcgaatatgatagtgaaaaaaatcctgtttcatcagtaaggaataggttacctgaaaaatatcaaactgaattgaacgatctgtacaatgaatttacccattataaaataaattctattaatgtacagaaattaggcgatataaaaactagaacacaatttaaaagtgatattaaaggtgtagattcgaatactatttcagcaggtgaggataatttatatataattttgaccgctttagtatcattacattattactttgactcaattgatagtaaaaaaacgattgaaagtatattattaattgacgaattggatgcaactttgcatccatcatttcaaataaaactattggaaaaaatgagagagtatgctcatgaatataaaattcaaatagtttttactacacacagtttatctgctttggaaaactctttgctaaataaagacaatgttatatatttgataaatcaagtaaccaatgttgatgtgatgtctgatccagatatttataaaataaaaatgcatttgaatactttgacaacaaacgatatttatcaagataaagttatacctgtttttaccgaggatgaggaagccaggtttctattagatagattgtttgaatattttcaaaatcaagaagaatttaaaaattttattgaagttaaacgattctttcatctagttaaagcaaatttgggagcaaatagtttaagagatatttttaaggataataaacttctaaagaataccatgaaagctatttgtatattagatggtgatcaaaatgtagataaagataaaatttatggcattattgctttacctggtaaaaatcattttaaaggagagacaggaaaatctccagaaaaacttttatttgattatgctgaagagttatttaataaggataataatttttggagtaataatacaattagcagtagaggctatagtaaagaatggtatcttgataaaataaaggttcctatagataaatttgaaaaagaagaaaagaatggtaaaaataaggaaaagaaacgccaatttaataaaaaattgtttaacgatcagcgagacttctttgatttactatttaaaaattggctaaataactctgaacatgagtcaatgattagaaaattttattatgatttaagggaaatgtttaaaaaagttgctccctataatggaatcgataaaaatctatggcaataa

SEQ ID NO: 7 - DNA, 1505 bp, L. johnsonii
atgaagactaatagtttagattttgatgcacaggctttaagagagaaaattttggacttggccatgcgcggaaagttggttaagcaagatccaaacgatgagccagcaagtgtattgcttgaaaagattaaggctgaaaaagcagaacttgttaaagagggcaagattaagaagagtaaggcccttcctgaaattactgatgaagaaaagccgtttgacatccccgatagttggaaatgggttagattgggggaaattggaaattatattcaacgaggaagatctccaaaatatgataaagaatctttagatcatcctataatttctcaaaaatgtgttcagtggaactcaattaatattgaagatgctaaacatataacagaagattttttcaaaaaattagaaccttatcgttttattcaaaaaaatgatatcctttggaattctacgggtacaggtactgttggtagattaaacattgtaaaagataactatagtcatgttcctgtggattctcacgttactcttatgagaatgaaccaattcatagattcagattatatctattattttttacgttcacctcaaattcaaaaaaatttaggagattatttaacaggaactactaaacaaaaagaattagggttaaccaacatattaaagataaatattccacttccacccgtttcagagcaaagtcgaatcgctgctaaaatagcgcagctattcgctttgcttcgcaaagtcgaatcttttactcaacaatacgcagaacttcagactttgctgaagtcgaaggtgttagatttagctatgcgcggaaaattggttaagcaagatccaaacgatgagccagcaagtgtattgcttgagaagattaaggctgagaaggaacagttgattaaagaaaagaagatcaaaaagagtaagccacttccgcagattagcgatgaagagaagccgtttgagattcctgatagttgggaatgggtaaggttgggggatgttggattaatagctacaggaaatactcctacgaaaaataatagtgatttttacggtggagctactccctttattaaacccgctgatattcaaaatttgattattaattataatacgaaggatttcctttctgaaaagggagaacaaaagggacgacttgcatataaaaatgatattttagttacatgtattggaaatttgggaagaaataacattattaataggaaagtcgcctataatcagcaaattaatggaatttctccgttaatagttagtagtagtttattgcattattttatgttaagttcattttttatatcttcaatgtataattatgcatccgccactacacttccaatattaaataaaactaaactttcaaatttaattattccacttccaccacttgctgaacaaagtggaatcgttgataaaatagaaatgatattttctttattttgcaaggatgaatcttctactcaacaattgaaaatctttaa

SEQ ID NO: 8 - DNA, 1434 bp, L. johnsonii
atgaaggtcattaaaaattacctttacaatgttggatatcaagtattagcaataattgtacctttaattacatcggcttatgtaagtcgggttttaagaccagaaggagtaggtgctaattcttttactaattccataattcaatattttattttatttgcaaatatgggaattggatattatggaaatagacaaatagcctatgtaagagaaaataaggatcaaatgtcgaaaactttttgggaaatccaaatagttaaaacaattatgactttatatgcttttgtagcatttgaaatatttatgatcttttatactcgtcaatctgagtatatgtgggctcaatcacttaatttaatagcgatcgcttttgatatttcatggttttatgaaggcatagaagattttaaagttacagtcttaaaaaattcattggtaaaagtaatatcgatgattgcaatatttgtctttataaagggacctaaggatgtgactttatatattattgtacttgcgttatcaacattaattggtaatttaacactatggccaaatataagacgtgatctcaataaggtttcttttaattttctaaatccgtggcaacactttttaccaatggcggaattgtttattccgcagattgcaacacaagtttatgttcagcttaataaaactatgctgggtgtaatggatagtgaaactgcatctggatattatcaatactctgataatttagtaaaattaattttggcattagttaccgcaaccggtactgttatgttgccacatgttgctaatgcggtttcacatggtgatatgcataaagtaaataaaatgctgtataagtcatttaattttgtctctgcagtttcttatcctatgatgtttgggttagctgctatttcacttactttagcacctaaatattatggttcgggctatggtcccgttggtcccgctatgatgattgaatctattgtgattttaattattgcatggagtaatgtattaggtgtgcagtatttattacctatccataaacaaaaacaatttacttggtctgttacgttaggagctgtcgttaatttaattctaaacgtaccattgatacggatttggggattaaatggagctatgtggtcaactgtattatctgaaatttcagtgactctatatcaaatgtgggcagtaagacatttgcttaattttaaagagttatttgcagattcatggaaatattttatagctggcttggtaatgtttgtacctgttttttggatgaatgcccatttgaaaaattcatggttaatgatggggatagaagttattgttggtgtaattgtttatatagtaattgtagctttactaaaagcttctataataaaggaagcaaaagaattagtagatcagaaattgcataaacataaataa

SEQ ID NO: 9 - DNA, 1407 bp, L. johnsonii
atgtggattaaacaaattgaatatgatggtggactagattataaaggaaaaataatttttgatagaaaaagtattattagtagtaaatataataaaaaggggaaaagtactttattaagagtaattctttatgcattgggatttgaagttgtaaggacaaatggaattgccaatttagacttaaaaacaaatataaaaattttaactgacgatttagaagagatacaaatttttagaaataacgaaaaagtaatactagaagaaagttctagtgatagaaaacgagaactacatttaccagaggatcaggaagtattgcaatctactttttttgattttaacaatggaaaattgttagaaaacttcttaggcactttttattttgaacaaggtagaggatattctttaattaataatggagtagttacaccgaaaaatcgttttaacttaaaagaattagtcacacaattagtacctgaagtagaagaaaagttaaaggaaatagagaaaaaagaaattacaattagaagaaataaagcagcaataaatgcaataaatgcagttcttaaggataacgataattttatagaagaagataatcaagatgaaataactattcaaaatataaatagtttaaagtttaaattaaaacagcttaaaagaaaaaagcgtacgattctggaatcgattgaagataataataaacttttagaatatattggtaaattaaaattacgattaagattatcagataaaagtgtagtagtagttacgtcagataaaatagaaggaatggaaaatatatcaaactatttaataagtgaaaaaacagtggttaataatcaaataaaagaaacaaaacagttaatatataaaaatgaagaggctattaagaaaaaatatacgaaccgagatattgcgaaattttctattccacaagcttttaattttgaaaaaaataaagctatgctgaaatatttaaataaacaagatgaagagcttgctttacaaaagaaagagttattaagaaaaaatagctatcaagaattacaagatagaatttataaattgtatactgaatatgcaagtgagctaaatatctcaaattggctaaaaaatggaattttctcaagtaaaaacatttcgggtcttacgggtacagaacaagaatttacggcaatatcaattcgattagcagctttgaaattaattgaggagtttacaaaagttaatttaccaattattttggattcaccatttcaagaattggatactaaaaacaaaaaattattagttgattttttagaaaatgaattttctgaaaaacaccaatttattataacttcagtgaataaagaaattcctaatgatatctataatcattggcaaattattactttggattaa

SEQ ID NO: 10 - DNA, 972 bp, L. johnsonii
atggttaagaaattctttaaaaataaaaaagctatatcttacaagtctattttttcattgcttttttatataaaaaaatctagtaaatatttgaaaagaaaaaaatttgataaattaattcttgaaaataatataccattggtatggacgatttatttatctcgttattcaggtgaatttttttatcattttcataatttgccacgcactactgcaaaagcggaagagattttaaagaagtgtacaggttttttatgtgtaagtgattatatttctaagcaattgataaatggaaatactcctcttggaaaaattgatcgaaataaggtacatactttatataattgtgtggatactaaaatgttttatccaatgcaaaatcgaaagaagtatagaaaagaaataaaaggaaaatacaagattcctgtagatgataaaattattctttttgttggacgaatttcgccagaaaaaggtcttaatattgttttagacgctattaataaaatgaaggtatctaattttaaattgctaattattggaagtgttatgcatggtaataatgaaaaagatgcgtatttttcaaaattagtatataattttgaaaaattaaaagataagataatatttactggttatgtttcccattcacagcttcctttattttataatgcagctgatttagtaattcttccttcaatgtgggaagaacctgcaggattaaccatggttgaaagtttagcttgtgggacccaattgattactacagattctggaggaattcctgaatatgtaaaagataagggtattattttaaagcgagataaagatcttgtagagaaaatagcagaaatatctgattcacttttagaaaaaacatatgaaaataaaatgagtaattttgaatatgtacaaaaatatttttctccgcataattacttaaaaaattttataaagattttaggtgaataa

SEQ ID NO: 11 - DNA, 1101 bp, L. johnsonii
atgataaaagatatatttagctttgttaatacacctcatcatgaagaatgttatatattacttggtattagagataatgatcataaaattataggcgtagaaaatgataataatagattaactactgacaaattaacaagttatataaataatttaccaattgctaatcactctgttcctaatattaaggttagaagtttaaaaatagataatcatgaaatagatgttattattattaaaaatagtgatgatgttcctgtgtttttaaatgaggagtatcatccaaaagaatgtaagaatgttattagaccaggtcaaattttttgtaggttaaatgatgttgaaacacctattaatggaactgcatcagattttcaagttgaaaggctttggaaaaaaagattccacttggatttaactccaattgaaatatacaaatctagattaaatgaaatagataattgggaatactttgaaactgataaagttggatttagatatattcttgatcccgattattgtatgtatttggaaagcaatgatgaagggcggaatattgtagaatcatattctctcaatcagacaagaattacgattaattgggatactttaaaactcatgtatcacggacagatattggaagaaattatggttgtttggcttgatggaacaagatttttaacagtggcgcccaatataggttctttgaatcctttgagtgacaagccattatattttcagtacttattaacggactcattagattttgcaatagaacaatttttcttaaataatagagaaagaggtattagtcctgatgcttttcagaaagatactttattaaaaaatatagtaatttttaaagacgaaattcagaaggaacaaatatgtaagttgcttgaagaagatcttgataccgttagaagttttgtgagaccttcatcagaccaattaaaatacgcaaaagctaagctcgaggcagaattgaatagggcacagctacctcagtttattaatgcggttgagcaaatgtgtacagaacaaaatactgcagattatataaaaaggtttattcaatcaaaagttagtaaagaaagataa

SEQ ID NO: 12 - DNA, 1185 bp, L. johnsonii
ttggtacataagaaagtaattaaagatcaaaaatatgaagattactggaagttaaccttaggaacatcagacttttatgggaatcaatttattagaacactagaaataattattaatcatattgatcaatatgatttaggaaataaaactgaagatgaattagtaattcgttcgaaaacaggaaaaaataatcgattaaatcaaaacgttactcactctaaagatttagagaataaaataaaagaaatttaccaaaatgatgattctagtggtgctacaacaagaaagcaaataaatcaatacattaaattaggttttattaaaccctattttcaaggctacgtaccagcagccaaagaatacataaagcctgggcaagataaagaaaaattaaaacggttattttctgataccgtttatacatatgcaagtttcaattcttctcaaacaaataaagatcaatttaatcagataaaatttttagtccaaacaattttgaatcgtaatagtaagaaattgaatgccttagaactcattggtttaattaatgattaccgaatacttacccaaacatacgcgaaagaaaaagatattgaaaatgacactcggtgggcaaaatctatttcttttgaggctagaaaatacaatcaaattagatatttatggaatatcttaagtaagatggcgctatttgaggtaagaggtagaaaaatggatgaacgggaagtaatactagctgaaaatgctaatgaattatttccggaacctggtagtactaagagagactcatatagatttgggttgatgaaaaaagctgtctacgaggaaagtattaagacttacggtaaaaaaatatgctggtttactaaaaaggaaaccatgggacttgtcgtttctcatttatatgcctctgcagatgctcttagaaactacgataccgatgcagcatatgatcccgaaaatgcattactactagcaccaggtaatccagatcaatatgtagataaatataaaatgacatttgactcaaacggacaaccaatttttgcaaaagatgctgatcctaattttattaatgaagcaaaaataaatggatatcatattgataaaaaaattatgaccagtgaaagacataaatatatgcaaaaacataatgctaaatttaaaataagaaataatagttaa

SEQ ID NO: 13 - DNA, 1131 bp, L. johnsonii
atgagtaggataaaggttttacatgttgcagaggcagctggtggcgtagaaagatatttggaaactttgtttaaatataatgataatagtaaagttgaaaatattttagtttgttcacaaaattatgattataaaagactgaaaaaattaactcaaaaaataactatattacaaatggagcatgaagtatctccgattaaagatataacagttgagagaaagttaagagatattataaaaaaagaaaaacctgatattgtctatgctcattccactaaagcaggtgcctttgcacgaattgcaaatttgggaataaataataagcttatatataatcctcatggttgggcttttaatatgcaacagtcaagcagaaaaaggcaaatgtataaatggtttgaaaaaatctcttctaatttttgtgataaaattatttgcatttcgaattcagagaaaaaatctgctttaagagagggaatttgtaaagcatcaaaattggaagttatttctaatggaatagatatagaaaaattgaaaagagttgagaaatcagctcgctcattaaaccaaataccaacaaatgcttttgtgattggacaggtagggcgattgtcaaaacagaaggctccagatatatttgtgagagcagctgaattaattaagaaaaaaatctctaattcttattttatcatggtgggagatggtgaattacgtgatgatattgagaaactaattagggataagaaattagaaaattcctttttaattactggttgggtagataaccctagtgagtatatgaaagttatggatgttggaacattattttcacggtgggaaggttttggattggttttgccagaatatatgctttgtaaagttcctgttgtagcaacacgtgttgatgcaattccgaatataattaaagatagcgttaatggattattggtgaattgcgataattatagagaagcagcttcagcaatagaaaacttatatgaggataaaaaactaaaaaacaatctagtaagaaatggatatagaatagctatccaaaactttgatggaaaagaaatgagtgctaaagtagagcaattatatatgaatcttataaagagggaaaatatatga

SEQ ID NO: 14 - DNA, 597 bp, L. johnsonii
ttgtatcgtattacacaaactcgagatccattttatttaagtattattttattaggttatcttacactttctaaaattacaggttataaattgcgattatacaaaattaaaatattttcttttatagaagaatatatatatttgataataccatgtatattaggaatgttattatttatatgtcctaaaaacattttttatatattaaatgatattttatcaaatagactagttttgtcacaaaatgctattgaaacatatggtattagaccttttggtacgtcaattcattttttaacgtcttataattggttaggacaaataacaggtcaatataattttatagatagcgcatatgtacagttattagtaggaaatggatatgttttcacatttttacttttatattttttaactgttataaataaaagagtaaggcaaaataatgatatatttttgctgggagtactttgtgtaattgctattcatgggatgtttgatccacaatttattttaccgtggtattctccgtttattttaatagctggcaaatattttatgacagaaacaaaagaaaagaaagagagtttgatttaa

SEQ ID NO: 15 - DNA, 1110 bp, L. johnsonii
atgaatactgatgaacttaaaaagtatattactcagagcattgatatggcgctaggttctgacatgcaaggcgagagtagttatactaacagtttttctattgagttagacaaaggtgggattaagtttattccgcggatgcccgcgggatacttaatcgatgataacttgtatcaacacatttttaagattttgaatgttgcactttatccgaactatacacttttgaaacaaaatacaacatactttgtaccgattagtacacgtaatattcatgttcaacgtgctctgtattttccatggcaagtgggaattccaagacgtttgatagttactaatcttgaggattttgcaacccagaatggcggtaagattcaaattatgaaaaatttgagtattgactataaccgtgtcacttcaattgctttggctggaacctctggtagtggcaagtcttatttcctaacctatctcttggaatccttaaagcatattgatgaaaatgtgcatggtgaaattggactcaaaactctaacagtaattgatcccaaaatggatgaacccagcagatgggctaaggctaatggcgttaaggttatttatcctgctacagatcgttcaaaatctgactttgtagcacaaattaatgatgtattaagtgaatgtttaaaattgatccacaagaggcaacaggtattatttaacaacccaagtactactttcaaacatgtaacagtggtaattgacgaagttttggcgctttcggaaggtattaataaaaatattaaagatacgttttttgctcttttatcacaaattgcattgttagggcgtgcaactcgtgtacacctaatattggtaagtcaacgttttgatcataatagcattccaacatcagtgcgcgaacaaatgaacgtattggttcagttgggcaatattaatcgtaaaacaacacagtttcttttcccagacctagatcccgaaggaattgtgattccattaggtcggggaactggtttgattcaggtcattgacgatgaacatccttatcaagttctgcccttattatgtccaacttattatttaagtagtaaggagaagattctatga

SEQ ID NO: 16 - DNA, 1101 bp, L. johnsonii
gtgaaaaaggcaattagagctagacaattcatgtacacccaagatcttgaccatctcccttttaaaaaggaaaagctaaaagatattctcgaaaagtcagatgccgaagaatgggcatatatactccatgataaagatattgacaaagatagaaaatctattagacctcattttcatgtagttctcaagtttaaggatgcaaagacaatttctcggatagctaaattatttactgataaacaagaatatattgaagtttggagaaatacgatcgggaatgcatatagctatttaattcatgaaactagtaatgccaaagataaacaccattatgatcctattgaagtagtatcttcatttgattttgaaactaaaatcaaacagattagaaaaaaagtagctaaaccttctaagaaagatatagacgacttaattgatgattattctaacgaaattttgacgaaaactaaacttcaagaaaaaattggtgttttagaaatggcaaaacacaagacacttttagatcacattgatgatattttagcttacaaaaaacatcaaaaatttcttaaagactttcaaggacaaaagtgcacaacttattggatttatggagaatcaggaatcggtaaaacaaaaatggtccgtgaaattcttgagcaacttcatccaaataattttgtagttcttggttcacaacgtgaccactttcaagaatatcgtggagaagaatttatagtcattaatgatttacgccctcgtgactatgaatacggtcaattgctgactttacttgatccttgggaaatcgataagatggcgccagcaagatatcatgataagtatctcaatgccagagctatctatattacaaccccttacagtccaatggctttttataatgaatcaggtattgtaaattctttgattgattcttttgatcaattaaatagaagaatcctatctctccatcttactagtgacacgtacaatcaaatgaaagctgacttaataaaagacgaccaaatagcagaagcaatatggaaaattaaaaagaaaaaaatcactagtcaaactgatagcgacaaaagtaatgactag

SEQ ID NO: 17 - DNA, 1077 bp, L. johnsonii
atgttacatgaagaaacttctcgatatatcactcaatgtttaaatatggcactagatctatcgggtgaaacaagctataccaacagcttcaaagtgatggtcaaatccgatggatttctattcattcccagaatgcccgcaagttatccacttgataatgatctgtatcaacggatttataaaattgctaatgcggcattatacccatataaatcattgttaaagcagtcaaccatgtatctagtagcaacagatgatattgattttggttcaaaaagagccttctactacccttggactggcgtaagtaaaagactccaaatctcggatatgaaatcatatcttgcttctgatactactaaagaaattcaaatcatgcaagatgtatccattgactataataaagtcacttcaatcttaattgctggtaatagtggatctggaaaatcatatgcattaacatacctgcttacgatgttacatttaaaaaatatttcggatctttatatcattgatccaaaatgtgacgtaccagcacgttgggctcatatttatggactagatgatagaactatttttccaactcaagaaatctcaaatagtgactttgtcaaccgatgtaatgagattttagctaaattagttaaaactatatacaacaggcaacggattttatacgaaaatccacatcatcaatttaaacatcttacagtttgcattgatgaggtgttggctcttacagatggtttgccgaaaaagattaaggatagtttcttttcattactatcacaaatatctcttttaggaagagcaacacgagttcatctcttattaataagccaacgtttcagcaatgacgctattccaatagctgttagagagcaagctaacgtttgcttgcaacttggtaacatcaacaggaaaacaactcaatttcttttcgatattgacccagatggcattttagtgcctacaggaaaaggaactggcttatttcaaataaccgacaatactcatccttttcaggtactacctttgttaacgcctacttataagattaaggacggtattttatga

SEQ ID NO: 18 - DNA, 1077 bp, L. johnsonii
atggttgaaaatcaacgggctaataaaactagagccggctttacgtttcaagatttagcagcaatagttttatttattgataattttgatgaattaaaaagtatcaaagcagagggaaataaagaagatattgacgtagaacttcttgatggtaattggatatatgctcaggctaagatgatcacagatccaattaattcaaaaaaagcatatagaagacgcagaatgagtgaatcgtttacgtcactttgtaataatttatttaagtctgaacagcctgtttctaatattgtctatatttcgaattgtaaagatccaataggtgaggaagatacacctactaaatctacttattctatttatccgttgaaatttaatcagcttgcaaatgttacacaaaaaagaatagaaaatatgttgggaaagaaaatagagagtgattatctaaaggaaaaaaacaaatcgaaagaagaagtattagatactttgaaaagaacatttggtgttcagataattccttttgatccacgatataaatatgaagataagtttaaatcggttcaaaattatgtaaacaatttcctaataagtagaaatttaatgcaaaatgctagcttagatattatgagaaattggcacgatttatttagagaaaatagtgaaagttattctaataataaagttttaactaagaaagatttgctttggattattgtagtaattgagtgtaatcaaccatttaatacagatgtattagcggctaaattaaaagttgatgtcgctattatggacggaataataatgagatttagggaagtttttgactatataagtgaacgattagaattttgttctagaatttggacagatgaagaagaatatattacaaataattcttcaaataatatatatgattttataaatagttcttggcagagttatgcgaatttatttaaagttgaaaatgtgtcggctgaggatcaagaaattttaactaaggtagcactgtatagagtgttgactaaaaaaaatacaattaagaatattatggaaacaggtaatgtgaatgtggattaa

SEQ ID NO: 19 - DNA, 1059 bp, L. johnsonii
atgaaaaaatatcaatttgtagaaacctatgctaatacacggaatgcggggagtaaggcacctcaagatatagaatttatagctaataaattgaaatttgaaccattaaaaattattcttggaaaagaaaaaggttcattttccaagataagtaatcagctatcttttattactcagtgggaaaaagcatataatgctatttcaagtaattctattgttttattgcagtatccgaagtatggtaggcagattaacagattgcattgtttaaagaaattgaaagaaagaaagaatgtacattttgtttctgtaattcatgatgttaatgagttgagaaatatagatcagaaaaaatctttaaggaatgaatttgaatttgtaattaaaaactcagattatgtcatactacataatgaatcaatgattgattattttgtaaaaagaggaatagatcctaaaaaattagttaatttacaaatttttgattatttgagaaataattatatgtctgattttccagtttttagtactacaatcaatattgcaggcaatttagatacaaacaagagtgagtatttaaagtatttagacaaagttgataataattttcaattgtttggaccaaatttttcattgaatagttattcgaatgttatatacgggggaagctttaaaccatctgaaattccaaatgtcttaaattccggatatggattaatatgggatggtacgagtatacacagctgtgagggtagttttggaaactatttaaaatataataatcctcataaattatctttatatttagcatctaatttacctgttattatttggagtaaagctgctgaagcaaattttgtaaaagaaaatggtatagggttgttaatcgacgatttgagtaaattacctaatattttgagtcagatatctaaagaagagtatttaacaatgtgttcgaatgtccaaaagattgctaaaaatttgtctaatggcttttatatgacaagtgctttaaatacagtaattactaaaatagagaataatttgaatgatgagtag

SEQ ID NO: 20 - DNA, 1053 bp, L. johnsonii
atggcaactattgaaaaacgtggctcttcatacagagctagagtaacaatttatcaacatggaaagcgtgattatttaaccaaaacatttaagacagaaaatgaagcgaaagtttggggaactactctagaacttgaaaaagcaaaaggtaaagctattgcacatcaaaacacactttttaaagatttctattacttatatgtgcataccatcaaagttaatgatgttagaaaagctacattcaataattatgtaaaagctggattagtaattaataacctctttcctactgccaaactaggaaaactagatgatgtacaaatgcaaaaaattttagataagtatggagaaacacattctaaaaagactgtagttgaattattaaagaaaattaggaccgctcttcgttatgcttacgcaaaaggatacatttataatgactttgctagtctgcttaaagctcatggtaaggaattacctaagcgtaataaagctttatcaatgtctgatttaactaagttaaaacattatttacttgaacatacagacgatgaatttaacctgatggttttacttgaaattagtactggtttacgacgcggtgaaattttaggtatcaaacccgaagatgtacattatgatggtcaatattactgcgttgaagtaagaaggtcaataagtcctactactgacgatactaaattaaaaactaaacattctagacgtagtgtaactattccaaaagatgtttatgacctattaaaaacaataccaagcaaagcttcaggctacctatttgattggtttagttttaagcaatctgagctacttaaaaaattactcaaaaaaataggtatcccctcaaccacatttcatggtttaagagatacccacgcttcttttttattcagtaataatattcctttatcctatgttagcaaaagattaggacatgattcaatcttaaccactgaaaaatattatctggaattaatgccagaaaaaaagcactcgcaagatgcgaatgccttaaatctattggcttccctatag

SEQ ID NO: 21 - DNA, 1044 bp, L. johnsonii
atgaaagtaatgattattggaatttgtagttatggaggaacaggctccagtgcagtagtcgatttattgaaagagtataaggaattacaggttcttggaaatgcggaatttcagtattcttttcaagtagatggattagaggacttagagtatcatctgcttaaacaatattctagacatatgtcaggtgatattgcaattcaccgctttttagactcaacaatttcgtgggctaaaactcccttagtgcataaaactattccacccaaggaatttataagattaacgaaaaaatatacaaattctttaatccaagaatgttggatagggcttgataattcagattatatgagtaaaagcattctgaaaaattctgttgttttaggatttaaaaaaataatttttcctttatatgaaaaaattactcatcattcttgggataaatggccagctagacgtttatatttgtcaattaatcctgatgatttttataaacttacgcaagagtatactactagtttgttgaaagctgctggagcagattttaataaaccagtagtacttgatcaaccttttgagggaaatgcaccgcaacaaagttttccatttttcaaagatcctagggctattgttgtggatagagatccacgtgatctttggattttagcaaaatatgctggtaactggactggtgaaggtcggtttatgcctagaaaagatgtaaaaacttttgtagagtattataaaaaattaagaaaaaatcagttaagagaagatagtgaaaatattatatttgtaaactttgaagatttgatttacgaatatgataagacaattgaaaaaattgaaaattttcttaatatttcacatcatgataatccgtataaatacttcaatcccaaaatttctataaataatacaagattaatggatagatatccgaatagtaaggccgatatggaatatattgaaaaaaatttatctgactatttgtatgatttctcaaaatataaaaaggttaaatatgatcgaaacattttttaa

SEQ ID NO: 22 - DNA, 954 bp, L. johnsonii
atgagaccaactatttcaattattataccagtatataatgttcaagattatttaacttcttgcatagagtcaataattaaacaagattataaggattacgaagcaatttttataaatgatggatctacggatgactctttagaaattttaaaacaatatgtaaagacggataaaagatttaaactaatttcgcaaaaaaattcaggattatcaagtgcacgtaatacgggcataaatcaggctaaaggcaaatatattacattcattgattcagatgactggattagcaaaaattatgtgggagctttagtttataatgctgaaaaatatgacgcagatatcgtttctattaaggaatgtttagtatatagtaatggaaaaaaagtttataaaaaacaagattttaaaattttaaagggaaaagcagcagatatgctgtttggattttatgatacaaattttgcatggggcaaattaattaaaacttcaattattaaagataataacatctattttcctgttggaaaaaattatgaagatataggaaccatgtataagatatatgataaagcacattgttcagtgcgcctttcaaatgaaaattatttttatcgttcacgtgaaagctcaattactgctacacgcagcataaaagatgtgaaagataaaatttcttttattaaagaaatgagaaagtattcgtcgttaagaaaaaagtataattattgggacttgtatctattagttaaagggtttggtgcaatatcagatgtatataaagtccctaatataacgaaaaaacaaagagatgttttggtaaaagaaataaaagttgcggtaaaaacttgtaaaattcatttaagagattttagagcggctgacggaatagatagagcccttttagtgaagattaatttggcggcattaattttaagaataaaacataaaggttag

SEQ ID NO: 23 - DNA, 885 bp, L. johnsonii
atgaattcgatccaggaacttaccgaagaaatttatactaaaattgctaatcgtgtgttaaaaagaaagcaaaaattaaaagttatgaattttcaaataattgatggatattgcaatagagaaaaattattatctagtattatgcataataagagaatacctaaaagaaatccgtatttattgaacgataaaatatcaaaatgtattgttaggaatcttaaattttcttcacaatatgaattagtgtggggaaaagacagtgaatatgattatcttatgcgggaggtatttgaaacaggggtcacatatttggaacaaagtacagaatatagcgatcttgttcataattgcttatatacctacttaccttttacaaaaatttttgctaagtatgagaattcgcttggacctgaaaagccagatgattcagctgtttttaattcacttgtgtcagctacagcatatgtatactattatgtttcggatgagataaaaaaaacacatcaagaagtctttttcgataaaggaacaaagaaattagataacagattagaaaaatattttgttgaggaaattccaaaagtattaaaaaaatatgttagtgatagccataataatggattagaaatatttaatatgttctcttcgattattaaatatgaaactgatgatcttatggaatctttagttaatggtccagaatggtatgcgcatcaaccagtaactaattcagatagaccatggtctgaaatgagagaaaaagttatagatgcaggggaaacctatattagcactttaatagaagaacagtctgagatggatccattcttttgtgacaacttacaagccgaaatcgatttggatgaagttttagatagtgaatag

SEQ ID NO: 24 - DNA, 870 bp, L. johnsonii
atgccaataactcaatctcctctacgttatccaggagggaaaacgcaattaactacatacgtaaagcatttattgaaggttaatggtatttcaggtacatatattgaaccttttgctggtggctttggtgtaggattagaacttttatattctaatgtggtttcaaatgttgtaataaatgacttggacccatcaatatttgcaatttggaatgctattttaaatcaaacagatgattttattcatttaatacaaacaacacccgtgaatatttatgaatggaaattacagcagaaaattagagaaaagacaaaaagtaatcctaattccattgataatggtttcgcttcttttttcttaaatagaacaaatgtaagtggaattattaatggtggtcctataggaggaaaaacgcagagtggaaaatataaattagattgtagatttaataaaaaggctctgattaaaaaaataaaaaaaattgcgtcacataaagacagaatcactttaaaaaatctggatgcaaataaatttatagtccaagagatacctaaatattctaagaaagatacttttatcttttttgatccgccttattacactcaaggtaaaaatctatatttatcttttgtaaataaagatgagcataaaaaattagctcaaaacattttaaatctatcagattataaatggataacgacatatgatatcgaagaggaaatcttaaaactctatcacccttatgttaagacttatacatatagattaaactattctgctaataagaaaagaaaagccaaggaatatatttttattaacaataatactaaagcagaatcatttgaaaaagtaaaattggatcgagtttga

SEQ ID NO: 25 - DNA, 852 bp, L. johnsonii
atggatatacagatgaacatacaacatggaaaggcttataaacagagacatcgacctatttgcggtatatatgcctttctaaatggaatcttacatgatgatgaccaaaaagaaaaacttcaaaaatgttatgtaaataaaatagcaaatcaaatatgggacatggctttaagttccataagtttgaataatacaaataaatataatcgtatccttgataagaatgatattttaaaggattattctttagttggggaattttatgattcggttactttagttaatttttttatacaaaaacatgactcaattataaagttgttacaacaatatagtttagacattaattatgaaattactgataattttaagcataagaatttagaactatatgatgatcaaataaaagaaaattgtttttatttaattcctataaattcgaatagaggatggggtaaaaataaaaacaatatgcattggatctgctataaaaagtgcgataaaaatcttgtcattttcaatagtggagatgaagtgacagaaaaaagagctaagagaaatgctgccttatgtaaaagtactatacgtagtaagtatattttaaatacttatgaattattagaagtatggaaaaatatgtataatcgtgagactgattatagaaataaaaagaacttagaattatattttgacttttacaaatggaaacctaagttattggtaaaattttttaaaaaacatactacgaaatatccttccaattattgggatagaattgataagataaacaaaggatgtaaatattcttttgagaaaagtaatttcaatatagtaaaagtaactattaaatga

SEQ ID NO: 26 - DNA, 795 bp, L. johnsonii
atgcaagtaagtactatagaaaatcgtgaagattatgtttcaatcaaagagttagttgatttaattgataaaaagtacatattgcacttacataacagtaaaaaagatgttgataaactggcaaaattagcacttgaaaagaaaataatttcgactattaagaaagaaaataatattgattatatccaagaaaatcgaggtaaaaaggtttatctaataaatcgaggaagcattaatgctttaatgaacttattagagaagtacatcattagtagacctcaatttacaaggcatgatgaggttctttatagtaaacaattagccaaaatcagtgatggaagtcaaaaggataaaactttaaaagaattgcaaattcagaaaagttccgactatctagaaggtaagagcctggaaaatgctaagaattgtattattcaaattattgataacaatttatataacacaacgtatgactctactcgaaaagttgttgagaaaatcgagaaatttgctgctgatagctacgacgattttgctgaggcttttaaaattcatttcaatcaagcttttgaggagaagctaacttatgatttaaacaaggtcaaaacggaaattatttttcaaaatagctttaagcaaaaatactcaagctttaatcaaattgcttatattaaagattattgtctgagagagcttcatacagtaaaatatgatgatactttcataattcgtaaagggtattcagagtatgacattaaattgcaaaatccacttacatggtactgcagaaaatag

SEQ ID NO: 27 - DNA, 822 bp, L. johnsonii
atgaaaaatcaacaaatccccaaagttataaattatttttggtttggtggaaaagatttacctcctaaagtaaaaaaatgcataaaaagttgggaaaagtattgtcctgattatgaattaaaattatggaatgagactaattttgatattaactgttgttcatatgttagagaagcctacgattcaaaaaaatgggcgtttgtaagcgactatgcgagattttgggttttatataattatggtggaatatatttagatacagacgttgaattgataaagtctttagataatattattaaaaatgggcctttttttgcattagaaactgaagaatatgatagtgtaaatccaggtgtaggaatggcttcacaaaagaaaaatgttttttataaagaagtattagatgattatgaaaactctcattatctaaccaaagacggaataccgaataatactcctgtgggaaaaagggttgcaaggctattgaaagaatatggtttaagcagtaataatagtgagggcattcaagtagtagatggtatttatatctatccagcagaatatttttgccctttgaactactttacaggagagttacatataacggataaaactgttgctattcatcattttcaagcatcgtggttaagtgaaagcgaaaaaaaataccataagattagtcaacatgtaaccaaactttttgggaaaagagcaggcgaaaagtttgaaatgattataaagtttccttattcatttaacaaaaagagaaaaaaattaggattaaaaacatctattaaatattatttgaggaaatataaataa

SEQ ID NO: 28 - DNA, 678 bp, L. johnsonii
atggaaaaagaaagctttgaagatcagtttaagaaactaactttatctgaaatagatgatattatatctaaatattccgattatccaatgaataaaactattagattacatgttttttatgatcagaataaggataagccgataaaaatttctgtaactaatcaagatagaaaaaatatgaatgagtttgccgagtttagacataatgcttatgcagcatattcaatttttgatattgaaaaacaagcttttgatttattgatgaatcagaagccggaatttccacctaaagctccagatattagtaataagctaatactagattatatttcagatgcaagggtctttatagattcaacaacaaattgggtgaagaaaaatattccagattactttgataaatgggacaatattagaaaagatttgtattcaacaagtgcgtcttacagaatatgttatcatttgagaaattatgtacagcataagatgtacgtaccaacagaagcagtatcagtattttcttctaatacagtagattacatgttggatatagataatttattggaagatagccagtttgtaaataaagtgaaattaccaaatgattattttaaagataagagtcatatgtacttaaaggggcatgtagttgaagagactgtcaaatcttttgtgtaa

SEQ ID NO: 29 - DNA, 480 bp, L. johnsonii
atgaatacacgagctgaaggaagatggtatgtgattaaaccttggcagtggggaatagattttacaaattggatatattacttaagctatggaatatttacctatagtgtgctgctaattaattatctaaaatgttggttttatcctatagcagctataatatgtgttttattactacttggagtagccatgttacttccttggaacagaaaaagcataaattatttaatatcatgtgaaatggccattatgacagctaatatgtttgatcataagttaatatatttatcaatgtatggttgtgtattattatcattgtatttgacagaacaattgcaaatatggattaaaagatttatagataaaacaacagcgcaagagcagagacagtatgtgaacagcattataagatactcgctgtctggttatggggctatagggatgataatattattcataattaccttactaaaacatgga

SEQ ID NO: 30 - DNA, 549 bp, L. johnsonii
gtggttattaacattctatcaaacaggtccgaatggactttttattttttatcaagtgttcaatttaattttacaatcagcgctaaaaatattttagataaatatatacctcttataataaaaaaatatatcgaaaatttagatgaagataatcgaaataagataataaaagcatattataatttagaatctttagttcaggattcgttatttttagaagatttaccgctagaacttgattttagtgaagatttaaagaagtttctaaaattagaaaatttgcatttagattctaagtttttaaaagagccctatgctataattgaatcgatcttaagaatccatcaaacatgtaatttgaagactgtgccagttgtctgtaatgtcgctcattatttagatagaacacagttaaacatgttgaacaatttactaaagtcaatgaaattgaaaatgattttaatagaattcacatctaaggatgttctggtttatccgaaagatgctgattacttctacattgatcaggaccttgttgattggtactga

SEQ ID NO: 31 - DNA, 387 bp, L. johnsonii
atgtatattgagaagtctgaggaactttataagtcaccgaatgatttatcgaataaattgattgagattcagaaaattgttataggaatgcataagcatacttatcctaaagagaaattatcgaataatgagatctataaatcgtttcaagatggcgatgtccctgagatagcagaaatagcagaaataggaagttattatccagagttgattccaagtataaaggaattagataaatcattttcagattattgtataaaattggatgatgttgcacatacgcctgaagaatcaattaaagaatttatggagaatgaaatggcaaactccagattagctcttgcaacagtaggaatagagttacaaaaattagttttaaaaaagtaa

SEQ ID NO: 32 - DNA, 531 bp, L. johnsonii
atgacaaaacccattgaatataagcctagtccatttaggctatactttccattaataatgttttgcagtagcttaacattaacactcgtaaatattggactgtatcatttttggaaatactcagatatttacctattaattacaaacgtaattaatattgttggattttttattattgtaatcattcaaatagttcgttccacctttattaaccacattaaatctctatttctaacttgggatatccatcacaagctaattattccaaagcaaattagtgattttacgactaagcccaagtcaagcccaaccaatgacatttataactcatacctgcgtcaaacgtatggagaatttgtacacaataacctgtatatctgtgttagaattcccaacaatattgaagttgctaaattactggatgataaattaatcaagttacgtgaaagcattgttaatcaatatcctaaatattctttcacaggctttgaacgtaaaggtgtttatctaattagtgtaggtacaaaataa

SEQ ID NO: 33 - DNA, 528 bp, L. johnsonii
atggcagagcttagacgcaataaaaatgacagtaatgattcatttaaattagcaatgtttggcataacgcaatatgatgaaattaagtttagaggaagaaaatacccagataactcttataagaacttaaaaaaatattgtcttgattatttgtatcttcaaaagtgtcaaacaagagcaattaatcatttacgatcattaattgagttatcttttcctgagttaaattcaatttttgttcatgcatcttcgataatggcattacaaatgtttagaatgtatgctcatcatgactttttagtcggcttaactgttaaaaatatggttgatagtctttatcgaataattgaaaaacatataaagcgaactttaattgagaaatactgtgctaaggttgggctagctgataaaggatcttatcctagtattgaagcagatgatcctacgatccttttaatttctgattattgtgatgaaatagaagcatataataatcgtttgataaaaggcaaaaagggaagtaattag

SEQ ID NO: 34 - DNA, 519 bp, L. johnsonii
atggatctaaatttttactatcaggatatcgttagagcttgcttacctaaagctcagatcgtcatcgatcgcttccacatggttcaaatgcttacccggtcgctcaactctttcagagtatccactatgaagagattcgacaagagatcgcatcaatatcaactgctcaaatctccctggaaactctatcttaaaaactacaaagacttggacaaggtacatccgcattacaattggcactacaaggattatcttactcaagagcagatcgtagcagaaggcattgagtgcagcgagagacttaaaagttcctacaacttaatgcaggacttcttccaagcattgaaaaatgaagatacagatatgcttaaaagagtcatccattccaaggcgggagtaggccctctaatgcataaaactctacttaccttcaaacataacctgagagctgtcttgaatggtgctactttcagctattctaacggctgcttagaaggcttcaatgtaagcgtcaaataa

SEQ ID NO: 35 - DNA, 306 bp, L. johnsonii
atgcagaagcagcaattaactgcattcatggaaaaaccgcttgatacccttgctaagggaagcgcagaatatatggaagcctataggtttaattcttatatcaggctatctaaagttaaattgaattggaataagattgaagtaaaagttcgaattcctgaatttcctgaaggtcaagcacaacttgatgcaatttgggataaagttgtcaagaaaatttatcgtatgaataatggagtttttactttatctaattataaaaatagtgaccctaattactacgttgtagaaggaacacgcgtttag

SEQ ID NO: 36 - DNA, 342 bp, L. johnsonii
atggaagatacaaaaccttttaaaacttatcgacaacaattatgcatacttagagcacgtggattagaagtcccgactgatgggaaagctaaacaggctctagaaagaataggctattatactttaattaaacatttgtttttagcaagagattcatatggtaatattattcatccagaaaagtttattccgggagttagtttcagagaaatagttgctttatatgatttagataaagaattaacgtctattctttatgatggactcgtagcttatgagataactttgggtgcagaaatttcatattttaactttgacggatgctatcaagccactacttag

SEQ ID NO: 37 - DNA, 336 bp, L. johnsonii
atgtgtgccttaaatcaatatgattttgaagcgtataataattttattgatggacatgcattaaataatacattagaaatattagccaaaagttcttcatttactacttattataaaaatttacttggttcagaatcacaaaagaatttaaatatgttaaatgaggttaataaaatttacaactttatatttaataattcaagtaatacttctacacttgttatttccgaagaagcaagcatagaatggccaacaggatacaaagatacgttaattaagtcctgtgctttattaagccctactgttaaatatgaagataaaataaataatgattaa

SEQ ID NO: 38 - DNA, 312 bp, L. johnsonii
atgaaaattagacgaaattcaggctactcaaaagaggtagcaaaaatgtacattcctagtgatgtacaaatcttttcactgtcaactgaactcaccaaacaggttaagtggattgatggtaaacccaccaatgaaataaatggttatcaagcttggtttgtcgcagaaggtactgaaccttttaaagttaaatttactaaaagagtcagtcttccaaaaatgttaaatcaaatttcattaacaaatcttgaggcttgtgaagttggatcaaatgtgtattttaaagcaacagatctcgaggtgattaactaa

SEQ ID NO: 39 - DNA, 207 bp, L. johnsonii
atgtatgaagagctatctactggtaaaaattttgaagaatcctggaattatactaagttagcttttaaagatacgcttttcaatcaagcgtatgatgtcatctcacatgaattaacagaagttgattttcaatttctctacgagatgtcaaaaataataatattagtgcaattatcgaagaaatgggtaaaagcaagcaatacgtaa

SEQ ID NO: 40 - DNA, 189 bp, L. johnsonii
atggctaaattacttactcgtgaagaagcttcaaagtacatcggtatcgatcctaaaacttttgataaagtttttcgtgctaatcctgattttaagagatttccactaagtgaccatagcgagcgttttactatcgaaagtattgaagacttcataaagcaaaaagaaacaactttaaagaaaatttga

SEQ ID NO: 41 - DNA, 156 bp, L. johnsonii
atgattcctaaaaaagcgactagtcgacccaggcttgtgttagaaagaccaccaactaataaggcaagagttctaaagattaaaatgctgcaaaatgctttagcaatattcttatcgtttgaattagacgatttaaagaaaacaattagatactga

SEQ ID NO: 42 - DNA, 147 bp, L. johnsonii
atgactcaaaagcaaactaatgaagaaaaatatattgatattcctttagatgacgatgttgaatgggacaaatatcctttggatgatgatgaaatttttgagtatcttatagctgaaaatgccgtaatgtatatggctaacaactag

SEQ ID NO: 43 - DNA, 147 bp, L. johnsonii
ttgctattaggctttactggcgatcttaatcgcttttcaacttataagcagttaaatgcttatcttggagtggatttaaacagatatcaatctgggaaatttgagaaaagagataagattaatcgtcgtggtagtagtcaagggtga

SEQ ID NO: 44 - DNA, 129 bp, L. johnsonii
atgatcctttcatctgggtctaagcggaataatggtaattgtattctccacgtatgtggagatgatcctcaagcagaggctgaatacggaattggtggtttagtgtattctccacgtatgtggagatga

SEQ ID NO: 45 - DNA, 126 bp, L. johnsonii
gtggatgaatgcactagagatatccaagaagataaagaagaagttttgcagtggatgaatgcactaaagatatccaaaaagattatgataaaaagtcttgcaacgaccaaaaaaaggtcgcactaa

SEQ ID NO: 46 - DNA, 120 bp, L. johnsonii
atggaaactattgcaccaattacacttgctactgcggcaatttgtgcagcttgtatagcactgtcaactgagccatgtgctaaatacatcatttcttcttacttatccttcctagactaa

SEQ ID NO: 47 - DNA, 117 bp, L. johnsonii
gtgcagcgttgttctgttggatcagcaagaagtgatctccacgtatgtggagatgatcctaatacgagaaacattaatactagtaaagaagtgtattctccacgtaagtggagatga

SEQ ID NO: 48 - DNA, 114 bp, L. johnsonii
atggattggcttaagaaggacagttttcatcatgatgatttaccaagaactacaatgtttttaggttttgttgcttcattgcttgcggcagtaatatctatatttaaaggttaa

SEQ ID NO: 49 - DNA, 114 bp, L. johnsonii
atggtctggctaacagacagtaagttctctgaagaatattttaattacttagaagcaaaagaaaatggtaatttagataagatctccaaattcaataaagcaaaaagccattaa